(19) United States
(12) Patent Application Publication
(10) Pub. No.: US 2008/0194454 A1
(43) Pub. Date: Aug. 14, 2008

(54) Perfume systems

(76) Inventors: George Kavin Morgan, Hamilton, OH (US); Zaiyou Liu, West Chester, OH (US); Robert Richard Dykstra, West Chester, OH (US); Alex Haejoon Chung, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 12/069,351

(22) Filed: Feb. 8, 2008

Related U.S. Application Data
(60) Provisional application No. 60/900,591, filed on Feb. 9, 2007.

(51) Int. Cl. A61K 8/00; A61Q 13/00

(52) U.S. Cl. 512/1

(57) ABSTRACT

The present invention relates to perfume systems, products comprising such systems and the use of same. Unfortunately current perfume systems do not always provide the desired sensory experience. Thus there is a need for one or more perfume systems that obviate the short comings of the current perfume technologies. The systems of the present invention meet the aforementioned need as they employ symbiotic combinations.

### CROSS-REFERENCES TO RELATED APPLICATIONS

This application claims priority under 35 U.S.C. §119(e) to U.S. Provisional Application Ser. No. 60/900,591 filed Feb. 9, 2007.

### BACKGROUND OF THE INVENTION

Products such as consumer products are typically designed and/or formulated to include a perfume system. The consumer who selects and uses such a product typically experiences at least three product moments of truth. The first moment of truth is typically at the point of purchase, the second moment of truth typically begins with the product's application and use, and the third moment of truth typically begins immediately after the product's application and use. Unfortunately current perfume systems do not provide the desired sensory experience during such moments of truth; as such systems do not provide the perfume level and balance at all three of such moments—especially the first moment of truth—that is desired. Furthermore, current perfume systems do not offer the economics and formulation flexibility that is desired. Thus there is a need for one or more perfume systems that obviate the short comings of the current perfume technologies. The systems of the present invention meet the aforementioned need.

### SUMMARY OF THE INVENTION

The present invention relates to perfume systems, products comprising such systems and the use of same.

### DETAILED DESCRIPTION OF THE INVENTION

### Definitions

As used herein “FMOT” means first moment of truth.

As used herein “SMOT” means second moment of truth.

As used herein “TMOT” means third moment of truth.

As used herein “PRM” means perfume raw material.

As used herein “perfume system” encompasses a single perfume technology and combinations of perfume technologies.

As used herein “consumer products” includes, unless otherwise indicated, articles, baby care, beauty care, fabric & home care, family care, feminine care, health care, snack and/or beverage products or devices intended to be used or consumed in the form in which it is sold, and is not intended for subsequent commercial manufacture or modification. Such products include but are not limited to diapers, bibs, wipes; products for and/or methods relating to treating hair (human, dog, and/or cat), including bleaching, coloring, dyeing, conditioning, shampooing, styling; deodorants and antiperspirants; personal cleansing products, including cleansers, moisturizing cleansers, and combinations thereof; cosmetics; skin care including application of creams, lotions, mousses, masks, exfoliating compositions, peels, and combinations thereof; hair removal products, including device-assisted hair removal products; shaving products; and other topically applied products for consumer use; products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee and cleaning and/or treatment compositions.

As used herein, the term “cleaning and/or treatment composition” includes, unless otherwise indicated, tablet, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types substrate-laden products such as dryer added sheets, dry and wetted wipes and pads, nonwoven substrates, and sponges; as well as sprays and mists.

As used herein, the term “fabric care composition” includes, unless otherwise indicated, fabric softening compositions, fabric enhancing compositions, fabric freshening compositions and combinations thereof.

As used herein, the term “solid” includes granular, powder, bar and tablet product forms.

As used herein, the term “situs” includes paper products, fabrics, garments, hard surfaces, hair and skin.

As used herein, the articles “a”, “an” and “the” when used in a claim, are understood to mean one or more of what is claimed or described.

As used herein, the terms “include”, “includes” and “including” are meant to be non-limiting.

Unless otherwise noted, all component or composition levels are in reference to the active level of that component or composition, and are exclusive of impurities, for example, residual solvents or by-products, which may be present in commercially available sources.

All percentages and ratios are calculated by weight unless otherwise indicated. All percentages and ratios are calculated based on the total packaged product, which includes the product and product matrix composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Consumer Product

The consumer who selects and uses a perfumed product makes critical decisions as to how satisfied he or she is with the product at multiple touch points in the product usage profile. Although numerous touch points are known, three product moments of truth are typically experienced by the consumer. The FMOT is typically at the point of purchase, the SMOT typically begins with the product's application and use, and the TMOT typically begins immediately after the product's application and use. Applicants have recognized that a consumer's FMOT is negatively impacted because the product packaging inhibits the sensory experience; for example, product packaging may make the product difficult to open or, when open, exposes a product that can spill. In addition, formulation ingredients can suppress and/or distort neat product odor. Furthermore Applicants have recognized that the consumer's SMOT is negatively impacted as volatile and high impact PRMs are lost during product storage, resulting in lack of bloom during use, where perfume bloom may occur initially upon product use, but does not last throughout the use experience. Compensating for these aforementioned deficiencies by adding high perfume levels for the TMOT can distort in-use scent experience, such that the perfume bloom can be too harsh or strong, and/or the perfume character can become less preferred. Also, Applicants have recognized that a consumer's FMOT was negatively impacted as perfume release from the treated situs, inter alia a dry fabric, over long period of time requires perfume levels in product that would distort the scent experience during the first and second moments of truth. Furthermore, addition of high perfume levels for SMOT & TMOT can distort neat product odor, and still not result in sufficient perfume deposition through the wash. In addition, perfume evaporation that occurs during drying can result in lower perfume levels on fabric; and/or the perfume remaining on dry fabric may provide initial dry fabric odor benefit but such perfume can dissipate too quickly to provide sufficient scent longevity benefits. Finally, Applicants recognized that solutions to the problems that are associated with one or two moments of truth can be insufficient to resolve the problems associated with the remaining moment(s) of truth or negatively impact the other moment(s) of truth. Thus, what is needed is a consumer product comprising a designed neat perfume and a perfume delivery system.

In one aspect, a consumer product comprising:
a.) from about 0.01% to about 20%, from about 0.03% to about 15%, from about 0.05% to about 10%, or from about 0.1% to about 5% of a neat perfume comprising, based on weight of said neat perfume:
   (i) from about 1% to about 30%, from about 2% to about 20%, from about 3% to about 15%, or from about 4% to about 10% of a perfume raw material having a boiling point less than or equal to 250° C. and a ClogP less than or equal to 2.5;
   (ii) from about 5% to about 70%, from about 10% to about 60%, from about 15% to about 50%, or from about 20% to about 40% of a perfume raw material having boiling point less than or equal to 250° C. and a ClogP greater than 2.5;
   (iii) from about 1% to about 30%, from about 2% to about 20%, from about 3% to about 15%, or from about 4% to about 10% of a perfume raw material having boiling point greater than 250° C. but less than or equal to 280° C.; and
b.) from about 0.01% to about 20%, from about 0.03% to about 15%, from about 0.5% to about 10%, or from about 0.1% to about 5% of a perfume delivery system; and
c.) the balance of said consumer product being a consumer product ingredient is disclosed.

In one aspect, when the perfume delivery system comprises a perfume microcapsule such microcapsule may comprise, based on total perfume microcapsule weight, from about 50% to about 95%, from about 60% to about 90%, from about 75% to about 85% perfume, at least 50%, 75%, 85% or even 100% said perfume being a perfume raw material having a ClogP greater than or equal to 1 and a boiling point less than or equal to 350° C.; a ClogP greater than or equal to 1.5 and a boiling point less than or equal to 300° C.; or even a ClogP greater than or equal to 2 and a boiling point less than or equal to 280° C.

In one aspect, when the perfume delivery system comprises a microcapsule, and an amine assisted delivery system, the neat perfume comprises:
(i) from about 2% to about 40%, from about 4% to about 30%, or from about 8% to about 20% of a perfume raw material having a boiling point less than or equal to 250° C. and a ClogP less than or equal to 2.5;
(ii) from about 4% to about 60%, from about 7% to about 50%, from about 10% to about 40%, or from about 15% to about 30% of a perfume raw material having boiling point less than or equal to 250° C. and a ClogP greater than 2.5; and
(iii) from about 1% to about 20%, from about 1% to about 15%, from about 2% to about 10%, or from about 3% to about 6% of a perfume raw material having boiling point greater than 250° C. but less than or equal to 280° C.

In one aspect, said amine assisted delivery system comprises a polyalkylamine.

In one aspect, said polyakylamine comprises a polyethyleneamine.

In one aspect, said polyethylenamine has a weight average molecular weight in daltons, as determined by dynamic light scattering (DLS) using a Malvern Zetasizer Nano-ZS, supplied by Malvern Instruments Ltd Enigma Business Park, Grovewood Road Malvern Worcestershire WR14 1XZ United Kingdom, of from about 500 to about 5,000,000, from about 1000 to about 1,000,000, or from about 7,000 to about 200,000.

In one aspect, when the consumer product is a packaged product that comprises a neat perfume, a perfume microcapsule, and a perfume delivery system that comprises a polymer assisted delivery matrix system comprising a perfume comprising:
(i) from 0% to 100%, from about 1% to about 30%, from about 2% to about 20%, from about 3% to about 15%, or from about 4% to about 10% of a perfume raw material having a boiling point less than or equal to 250° C. and a ClogP less than or equal to 2.5;
(ii) from 0% to 100%, from about 5% to about 70%, from about 10% to about 60%, from about 15% to about 50%, or from about 20% to about 40% of a perfume raw material having boiling point less than or equal to 250° C. and a ClogP greater than 2.5; and
(iii) from about 0% to about 100%, from about 1% to about 30%, from about 2% to about 20%, from about 3% to about 15%, or from about 4% to about 10% of a perfume raw material having boiling point greater than 250° C. but less than or equal to 280° C.;
   said neat perfume comprises:
(i) from about 1% to about 30%, from about 2% to about 20%, from about 3% to about 15%, or from about 4% to about 10% of a perfume raw material having a boiling point less than or equal to 250° C. and a ClogP less than or equal to 2.5;
(ii) from about 10% to about 90%, from about 15% to about 80%, from about 20% to about 70%, or from about 25% to about 60% of a perfume raw material having boiling point less than or equal to 250° C. and a ClogP greater than 2.5; and
(iii) from about 1% to about 30%, from about 2% to about 20%, from about 3% to about 15%, or from about 4% to about 10% of a perfume raw material having boiling point greater than 250° C. but less than or equal to 280° C.

In one aspect, said polymer assisted delivery matrix system is in either in whole or in part, (for example, adhered to) in communication with the packaged product's packaging and/or the exterior of the packaged product's container, for example any cap that may cap or be attached to said container. In one aspect, said polymer assisted delivery matrix system is either in whole or in part in communication with the underside of the aforementioned cap.

In one aspect, when the consumer product is a packaged product that comprises a neat perfume, a perfume microcapsule, an amine assisted delivery system and a perfume delivery system that comprises a polymer assisted delivery matrix system said neat perfume comprises:
(i) from about 2% to about 40%, from about 4% to about 30%, or from about 8% to about 20% of a perfume raw material having a boiling point less than or equal to 250° C. and a ClogP less than or equal to 2.5;
(ii) from about 4% to about 60%, from about 7% to about 50%, from about 10% to about 40%, or from about 15% to about 30% of a perfume raw material having boiling point less than or equal to 250° C. and a ClogP greater than 2.5; and
(iv) from about 1% to about 20%, from about 1% to about 15%, from about 2% to about 10%, or from about 3% to about 6% of a perfume raw material having boiling point greater than 250° C. but less than or equal to 280° C.

In one aspect, suitable perfume raw materials having a boiling point less than or equal to 250° C. and a ClogP less than or equal to 2.5 are those materials listed in Table 1 below and such materials are defined as Table 1 perfume raw materials.

TABLE 1

| Number | Registry Name | Trade Name |
|---|---|---|
| 1 | Propanoic acid, ethyl ester | Ethyl Propionate |
| 2 | Acetic acid, 2-methylpropyl ester | Isobutyl Acetate |
| 3 | Butanoic acid, ethyl ester | Ethyl Butyrate |
| 4 | Butanoic acid, 2-methyl-, ethyl ester | Ethyl-2-Methyl Butyrate |
| 5 | 2-Hexenal, (E)- | 2-Hexenal |
| 6 | 1-Butanol, 3-methyl-, acetate | Iso Amyl-Acetate |
| 7 | 2-Buten-1-ol, 3-methyl-, acetate | Prenyl Acetate |
| 8 | 2-Hexen-1-ol | Beta Gamma Hexenol |
| 9 | 3-Hexen-1-ol | Beta Gamma Hexenol |
| 10 | Benzaldehyde | Benzaldehyde |
| 11 | 3-Hexen-1-ol, acetate, (Z)- | Cis 3 Hexenyl Acetate |
| 12 | Benzoic acid, methyl ester | Methyl Benzoate |
| 13 | Benzeneacetaldehyde | Phenyl Acetaldehyde |
| 14 | Benzeneacetic acid, methyl ester | Methyl Phenyl Acetate |
| 15 | 1,3-Dioxolane-2-acetic acid, 2-methyl-, ethyl | Fructone |
| | ester | |
| 16 | Benzeneacetaldehyde, .alpha.-methyl- | Hydratropic Aldehyde |
| 17 | 3-Cyclohexene-1-carboxaldehyde, 3,5- | Cyclal C, |
| | dimethyl- | |
| 18 | Acetic acid, (2-methylbutoxy)-, 2-propenyl ester | Allyl Amyl Glycolate |
| 19 | Benzenemethanol, .alpha.-methyl-, acetate | Methyl Phenyl Carbinyl Acetate |
| 20 | Acetic acid, (3-methylbutoxy)-, 2-propenyl ester | Allyl Amyl Glycolate |
| 21 | Benzaldehyde, 4-methoxy- | Anisic Aldehyde |
| 22 | Benzeneacetic acid, ethyl ester | Ethyl Phenyl Acetate |
| 23 | 2-Cyclohexen-1-one, 2-methyl-5-(1- | Laevo Carvone |
| | methylethenyl)-, (R)- | |
| 24 | Ethanol, 2,2′-oxybis- | Calone 161 |
| 25 | Acetic acid, 2-phenylethyl ester | Phenyl Ethyl Acetate |
| 26 | Benzoic acid, 2-amino-, methyl ester | Methyl Anthranilate |
| 27 | 4,7-Methano-1H-inden-6-ol, 3a,4,5,6,7,7a- | Flor Acetate |
| | hexahydro-, acetate | |
| 28 | Octanal, 7-hydroxy-3,7-dimethyl- | Hydroxycitronellal |
| 29 | 2(3H)-Furanone, 5-ethyldihydro- | Gamma Hexalactone |
| 30 | Phenol, 4-methyl- | Para Cresol |
| 31 | Bicyclo[2.2.1]heptan-2-one, 1,7,7-trimethyl-, | Camphor Gum |
| | (1R)- | |
| 32 | 2H-Pyran, 3,6-dihydro-4-methyl-2-(2-methyl-1- | Nerol Oxide |
| | propenyl)- | |
| 33 | Benzeneethanol, .beta.-methyl- | Hydratropic Alcohol |
| 34 | Benzeneethanol, .alpha.,.alpha.-dimethyl- | Dimethyl Benzyl Carbinol |
| 35 | Benzoic acid, 2-(methylamino)-, methyl ester | Dimethyl Anthranilate |
| 36 | 2-Propenal, 3-phenyl- | Cinnamic Aldehyde |
| 37 | 2-Propenoic acid, 3-phenyl-, methyl ester | Methyl Cinnamate |
| 38 | 4H-Pyran-4-one, 2-ethyl-3-hydroxy- | Ethyl Maltol |
| 39 | Acetic acid ethyl ester | Ethyl Acetate |
| 40 | 2-Heptanone | Methyl Amyl Ketone |
| 41 | Acetic acid, pentyl ester | Iso Amyl-Acetate |
| 42 | 3-Octanone | Ethyl Amyl Ketone |
| 43 | 2-Octanone | Methyl Hexyl Ketone |
| 44 | Heptenone, methyl- | Methyl Heptenone |
| 45 | 1-Heptanol | Heptyl Alcohol |
| 46 | 5-Hepten-2-one, 6-methyl- | Methyl Heptenone |
| 47 | Butanoic acid, 3-oxo-, ethyl ester | Ethyl Acetoacetate |
| 48 | Ethanol, 2-(2-methoxyethoxy)- | Veramoss Sps |
| 49 | Tricyclo[2.2.1.02,6]heptane, 1-ethyl-3- | Neoproxen |
| | methoxy- | |
| 50 | Benzene, 1,4-dimethoxy- | Hydroquinone Dimethyl Ether |
| 51 | Carbonic acid, 3-hexenyl methyl ester, (Z)- | Liffarome |
| 52 | Oxirane, 2,2-dimethyl-3-(3-methyl-2,4- | Myroxide |
| | pentadienyl)- | |
| 53 | Ethanol, 2-(2-ethoxyethoxy)- | Diethylene Glycol Mono |
| | | Ethylether |
| 54 | Cyclohexaneethanol | Cyclohexyl Ethyl Alcohol |
| 55 | 3-Octen-1-ol, (Z)- | Octenol Dix |
| 56 | 3-Cyclohexene-1-carboxaldehyde, 3,6- | Cyclovertal |
| | dimethyl- | |
| 57 | 1,3-Oxathiane, 2-methyl-4-propyl-, cis- | Oxane |
| 58 | Acetic acid, 4-methylphenyl ester | Para Cresyl Acetate |
| 59 | Benzene, (2,2-dimethoxyethyl)- | Phenyl Acetaldehyde Dimethyl |
| | | Acetal |
| 60 | Ethanone, 1-(4-methylphenyl)- | Para Methyl Acetophenone |
| 61 | Propanoic acid, phenylmethyl ester | Benzyl Propionate |
| 62 | Octanal, 7-methoxy-3,7-dimethyl- | Methoxycitronellal Pq |
| 63 | Linalool oxide | Linalool Oxide |
| 64 | 2H-1-Benzopyran-2-one, octahydro- | Octahydro Coumarin |
| 65 | Benzenepropanal, .beta.-methyl- | Trifernal |
| 66 | 4,7-Methano-1H-indenecarboxaldehyde, | Formyltricyclodecan |
| | octahydro- | |
| 67 | 2-Butanone, 4-phenyl- | Benzyl Acetone |
| 68 | Ethanone, 1-(4-methoxyphenyl)- | Para Methoxy Acetophenone |
| 69 | Benzoic acid, 2-hydroxy-, methyl ester | Methyl Salicylate USP |
| 70 | Propanenitrile, 3-(3-hexenyloxy)-, (Z)- | Parmanyl |
| 71 | 1,4-Methanonaphthalen-5(1H)-one, | Tamisone |
| | 4,4a,6,7,8,8a-hexahydro- | |
| 72 | Benzene, [2-(2-propenyloxy)ethyl]- | LRA 220 |
| 73 | Benzenepropanol | Phenyl Propyl Alcohol |
| 74 | Ethanol, 2-phenoxy- | Phenoxyethanol |
| 75 | 1H-Indole | Indole |
| 76 | 1,3-Dioxolane, 2-(phenylmethyl)- | Ethylene Glycol Acetal/Phenyl |
| | | Acetaldehy |
| 77 | 2H-1-Benzopyran-2-one, 3,4-dihydro- | Dihydrocoumarin |

In one aspect, suitable Table 1 perfume raw materials include perfume raw materials from number 1 to number 39 and mixtures thereof.

In one aspect, suitable Table 1 perfume raw materials include perfume raw materials from number 1 to number 29 and mixtures thereof.

In one aspect, suitable perfume raw materials having boiling point less than or equal to 250° C. and a ClogP greater than 2.5 are those materials listed in Table 2 below and such materials are defined as Table 2 perfume raw materials.

TABLE 2

| Number | Registry Name | Trade Name |
|---|---|---|
| 1 | Bicyclo[2.2.1]heptane, 2,2-dimethyl-3- | Camphene |
| | methylene- | |
| 2 | Bicyclo[3.1.1]heptane, 6,6-dimethyl-2- | Beta Pinene |
| | methylene-, (1S)- | |
| 3 | Bicyclo[3.1.1]hept-2-ene, 2,6,6- | Alpha Pinene |
| | trimethyl- | |
| 4 | Propanoic acid, pentyl ester | Amyl Propionate |
| 5 | 1,6-Octadiene, 7-methyl-3-methylene- | Myrcene |
| 6 | Cyclohexene, 1-methyl-4-(1- | Dipentene |
| | methylethenyl)- | |
| 7 | Cyclohexene, 1-methyl-4-(1- | Terpineolene |
| | methylethenyl)- | |
| 8 | Acetic acid, hexyl ester | Hexyl Acetate |
| 9 | Cyclohexene, 1-methyl-4-(1- | Terpineolene |
| | methylethylidene)- | |
| 10 | Benzene, 1-methoxy-4-methyl- | Para Cresyl Methyl Ether |
| 11 | 1-Octen-3-ol, acetate | Amyl Vinyl Carbinyl Acetate |
| 12 | Octanal | Octyl Aldehyde |
| 13 | 2-Oxabicyclo[2.2.2]octane, 1,3,3- | Eucalyptol |
| | trimethyl- | |
| 14 | Butanoic acid, pentyl ester | Amyl Butyrate |
| 15 | Heptanoic acid, ethyl ester | Ethyl Oenanthate |
| 16 | 5-Heptenal, 2,6-dimethyl- | Melonal |
| 17 | Hexanoic acid, 2-propenyl ester | Allyl Caproate |
| 18 | 3-Cyclohexene-1-carboxaldehyde, | Ligustral |
| | dimethyl- | |
| 19 | 3-Hexene, 1-(1-ethoxyethoxy)-, (Z)- | Leaf Acetal |
| 20 | Octanal, 3,7-dimethyl- | Dihydrocitronellal |
| 21 | 2-Octynoic acid, methyl ester | Methyl Heptine Carbonate |
| 22 | 2-Nonenal | 2 Nonen-1-Al |
| 23 | 1,6-Octadien-3-ol, 3,7-dimethyl- | Linalool |
| 24 | Benzoic acid, ethyl ester | Ethyl Benzoate |
| 25 | 6-Octenal, 3,7-dimethyl- | Citronellal |
| 26 | Cyclohexanol, 1-methyl-4-(1- | Dihydroterpineol |
| | methylethyl)- | |
| 27 | 1-Hexanol, 3,5,5-trimethyl-, acetate | Iso Nonyl Acetate |
| 28 | 3,5-Octadien-2-ol, 2,6-dimethyl-, (?,Z)- | Muguol |
| 29 | Cyclohexanone, 5-methyl-2-(1- | Iso Menthone |
| | methylethyl)-, cis- | |
| 30 | Heptanoic acid, 2-propenyl ester | Allyl Heptoate |
| 31 | Butanoic acid, 3-hexenyl ester, (Z)- | Cis 3 Hexenyl Butyrate |
| 32 | 1,6-Octadien-3-ol, 3,7-dimethyl-, | Linalyl Formate |
| | formate | |
| 33 | 3-Cyclohexen-1-ol, 4-methyl-1-(1- | Terpinenol |
| | methylethyl)- | |
| 34 | Bicyclo[2.2.1]heptan-2-ol, 1,3,3- | Fenchyl Alcohol |
| | trimethyl- | |
| 35 | Cyclohexanol, 2-(1,1-dimethylethyl)-, | Verdol |
| | cis- | |
| 36 | 3-Octanol, 3,7-dimethyl-, acetate | Tetrahydro Linayl Acetate |
| 37 | Bicyclo[2.2.1]heptan-2-ol, 1,7,7- | Borneol Crystals |
| | trimethyl-, (1S-endo)- | |
| 38 | Decanal | Decyl Aldehyde |
| 39 | 3-Cyclohexene-1-methanol, | Alpha Terpineol |
| | .alpha.,.alpha.,4-trimethyl- | |
| 40 | Cyclohexanol, 5-methyl-2-(1- | Menthol |
| | methylethyl)- | |
| 41 | 3-Cyclohexene-1-carboxaldehyde, | Iso Cyclo Citral |
| | 2,4,6-trimethyl- | |
| 42 | 7-Octen-2-ol, 2,6-dimethyl-, acetate | Dihydro Terpinyl Acetate |
| 43 | 2H-Pyran-2-one, 6-butyltetrahydro- | Nonalactone |
| 44 | 3-Hepten-2-one, 3,4,5,6,6-pentamethyl- | Koavone |
| 45 | 1,6-Nonadien-3-ol, 3,7-dimethyl- | Ethyl Linalool |
| 46 | 4-Decenal, (E)- | Decenal (Trans-4) |
| 47 | Terpineol | Terpineol |
| 48 | 7-Octen-2-ol, 2-methyl-6-methylene-, | Myrcenyl Acetate |
| | acetate | |
| 49 | 2-Butenoic acid, 2-methyl-, 3-hexenyl | Cis-3-Hexenyl Tiglate |
| | ester, (E,Z)- | |
| 50 | 1,6-Octadien-3-ol, 3,7-dimethyl-, | Linalyl Acetate |
| | acetate | |
| 51 | Benzene, 1-methoxy-4-(1-propenyl)-, | Anethol Usp |
| | (E)- | |
| 52 | 2-Decenal | 2 Decene-1-Al |
| 53 | 2,6-Octadienal, 3,7-dimethyl- | Citral |
| 54 | 6-Octen-1-ol, 3,7-dimethyl-, formate | Citronellyl Formate |
| 55 | Cyclopentanone, 3-methyl-2-pentyl- | Jasmylone |
| 56 | Undecenal | Iso C-11 Aldehyde |
| 57 | 6-Octen-1-ol, 3,7-dimethyl- | Citronellol |
| 58 | Cyclohexanemethanol, | Dihydro Terpinyl Acetate |
| | .alpha.,.alpha.,4-trimethyl-, acetate | |
| 59 | 3-Cyclohexene-1-methanol, | Terpinyl Acetate |
| | .alpha.,.alpha.,4-trimethyl-, acetate | |
| 60 | 2,6-Octadien-1-ol, 3,7-dimethyl-, | Geranyl Formate |
| | formate, (E)- | |
| 61 | Bicyclo[2.2.1]heptan-2-ol, 1,3,3- | Fenchyl Acetate |
| | trimethyl-, acetate | |
| 62 | Bicyclo[2.2.1]heptan-2-ol, 1,7,7- | Iso Bornyl Acetate |
| | trimethyl-, acetate, exo- | |
| 63 | 2,6-Octadien-1-ol, 3,7-dimethyl-, (E)- | Geraniol |
| 64 | 2,6-Octadien-1-ol, 3,7-dimethyl-, (Z)- | Nerol |
| 65 | Cyclohexanol, 2-(1,1-dimethylethyl)-, | Verdox |
| | acetate | |
| 66 | Undecanal, 2-methyl- | Methyl Nonyl Acetaldehyde |
| 67 | Undecanal | Undecyl Aldehyde |
| 68 | 2H-Pyran-2-one, tetrahydro-6-pentyl- | Delta Decalactone |
| 69 | 6-Octen-1-ol, 3,7-dimethyl-, acetate | Citronellyl Acetate |
| 70 | 10-Undecenal | Intreleven Aldehyde Sp |
| 71 | 2(3H)-Furanone, 5-hexyldihydro- | Gamma Decalactone |
| 72 | 2,6-Octadien-1-ol, 3,7-dimethyl-, | Geranyl Acetate |
| | acetate, (E)- | |
| 73 | 2H-Pyran-2-one, tetrahydro-6-(3- | Jasmolactone |
| | pentenyl)- | |
| 74 | Cyclohexanol, 5-methyl-2-(1- | Menthyl Acetate |
| | methylethyl)-, | |
| | acetate, (1.alpha.,2.beta.,5.alpha.)- | |
| 75 | 2-Undecenal | 2-Undecene-1-Al |
| 76 | 2H-Pyran-2-one, tetrahydro-6-(2- | Jasmolactone |
| | pentenyl)-, (Z)- | |
| 77 | 2,6-Octadien-1-ol, 3,7-dimethyl-, | Neryl Acetate |
| | acetate, (Z)- | |
| 78 | Benzeneethanol, .alpha.,.alpha.- | Dimethyl Benzyl Carbinyl Acetate |
| | dimethyl-, acetate | |
| 79 | 4,9-Decadienal, 4,8-dimethyl- | Floral Super |
| 80 | 3-Octanol | Octanol-3 |
| 81 | 2-Heptanol, 2,6-dimethyl- | Dimethyl-2, 6-Heptan-2-Ol |
| 82 | Propanoic acid, 2-methyl-, 1,3- | Iso Pentyrate |
| | dimethyl-3-butenyl ester | |
| 83 | 3-Nonanone | Ethyl Hexyl Ketone |
| 84 | 2,4,6-Octatriene, 2,6-dimethyl- | Allo-Ocimene |
| 85 | Bicyclo[2.2.1]heptane, 2-ethyl-5- | Neoproxen |
| | methoxy- | |
| 86 | 1-Octanol | Octyl Alcohol |
| 87 | 3-Octanol, 3,7-dimethyl- | Linacsol |
| 88 | Propanoic acid, 2-methyl-, 3-hexenyl | Verdural B Extra |
| | ester, (Z)- | |
| 89 | 2H-Pyran, tetrahydro-4-methyl-2-(2- | Methyl Iso Butenyl Tetrahydro Pyran |
| | methyl-1-propenyl)- | |
| 90 | Nonanal | Nonyl Aldehyde |
| 91 | Hexanoic acid, 2-methylpropyl ester | Iso Butyl Caproate |
| 92 | Cyclohexane, 3-ethoxy-1,1,5-trimethyl- | Herbavert |
| 93 | 7-Octen-2-ol, 2-methyl-6-methylene-, | Dihydro Myrcenol |
| | dihydro deriv. | |
| 94 | Ethanone, 1-(3,3-dimethylcyclohexyl)- | Herbac |
| 95 | Propanoic acid, 2,2-dimethyl-, hexyl | Hexyl Neo Pentanoate |
| | ester | |
| 96 | 3-Heptanone, 5-methyl-, oxime | Stemone |
| 97 | Isononanol | Iso Nonyl Alcohol |
| 98 | Cyclohexanone, 2-(1-methylpropyl)- | 2-Sec-Butyl Cyclo Hexanone |
| 99 | Butanoic acid, 2-methyl-, hexyl ester | Hexyl-2-Methyl Butyrate |
| 100 | 1-Nonanol | Nonyl Alcohol |
| 101 | Cyclohexaneethanol, acetate | Cyclohexyl Ethyl Acetate |
| 102 | 1-Octanol, 3,7-dimethyl- | Dimethyl Octanol |
| 103 | Cyclopentanone, 2-pentyl- | Delphone |
| 104 | Cyclohexanemethanol, 4-(1- | Mayol |
| | methylethyl)-, cis- | |
| 105 | 6-Octen-1-ol, 3,7-dimethyl-, (S)- | Baranol |
| 106 | Benzaldehyde, 4-(1-methylethyl)- | Cuminic Aldehyde |
| 107 | Propanoic acid, 2-methyl-, | Benzyl Iso Butyrate |
| | phenylmethyl ester | |
| 108 | Propanoic acid, 2-methyl-, 4- | Para Cresyl Iso Butyrate |
| | methylphenyl ester | |
| 109 | Carbonic acid, 4-cycloocten-1-yl | Violiff |
| | methyl ester | |
| 110 | 1,6-Octadien-3-ol, 3,7-dimethyl-, | Linalyl Propionate |
| | propanoate | |
| 111 | Cyclohexanemethanol, .alpha.-methyl- | Mugetanol |
| | 4-(1-methylethyl)- | |
| 112 | Butanoic acid, phenylmethyl ester | Benzyl Butyrate |
| 113 | 4,7-Methano-1H-inden-5-ol, octahydro-, | Dihydro Cyclacet |
| | acetate | |
| 114 | 2-Cyclopenten-1-one, 3-methyl-2- | Dihydrojasmone |
| | pentyl- | |
| 115 | Bicyclo[2.2.1]heptan-2-ol, 1,7,7- | Iso Bornyl Propionate |
| | trimethyl-, propanoate, exo- | |
| 116 | 2,6-Octadienenitrile, 3,7-dimethyl- | Geranyl Nitrile |
| 117 | Benzene, ethenyl- | Styrene |
| 118 | Benzene, methyl(1-methylethyl)- | Cymene Coeur |
| 119 | Cyclohexanol, 3,3,5-trimethyl-, cis- | Trimethylcyclohexanol |
| 120 | 1-Hexanol, 5-methyl-2-(1- | Tetrahydro Lavandulol |
| | methylethyl)-, (R)- | |
| 121 | Cyclohexanol, 4-(1-methylethyl)- | Roselea |
| 122 | 7-Octen-2-ol, 2,6-dimethyl-, formate | Dimyrcetol |
| 123 | Cyclohexanone, 5-methyl-2-(1- | Menthone Racemic |
| | methylethyl)-, trans- | |
| 124 | 1,3,5-Undecatriene | Galbanolene Super |
| 125 | 5,7-Octadien-2-ol, 2,6-dimethyl- | Ocimenol |
| 126 | 2-Cyclohexene-1-carboxylic acid, | Methyl Cyclogeranate |
| | 2,6,6-trimethyl-, methyl ester | |
| 127 | Benzene, (2-bromoethenyl)- | Brom Styrol |
| 128 | Benzene, 1-methoxy-4-(2-propenyl)- | Methyl Chavicol |
| 129 | 2H-Pyran, 6-butyl-3,6-dihydro-2,4- | Gyrane |
| | dimethyl- | |
| 130 | Cyclohexanemethanol, .alpha.,3,3- | Aphermate |
| | trimethyl-, formate | |
| 131 | Cyclohexanol, 4-(1,1-dimethylethyl)- | Patchon |
| 132 | Cyclohexanol, 5-methyl-2-(1- | Menthol Natural |
| | methylethyl)-, [1R- | |
| | (1.alpha.,2.beta.,5.alpha.)]- | |
| 133 | 1,3-Dioxane, 2-butyl-4,4,6-trimethyl- | Herboxane |
| 134 | 2-Nonynoic acid, methyl ester | Methyl Octine Carbonate |
| 135 | 6-Octenenitrile, 3,7-dimethyl- | Baranyl Nitrile |
| 136 | Decanal, 2-methyl- | Methyl Octyl Acetaldehyde |
| 137 | 2-Nonanol, 6,8-dimethyl- | Nonadyl |
| 138 | Phenol, 4-(1,1-dimethylethyl)- | Para Tertiary Butyl Phenol |
| 139 | 1-Hexanol, 5-methyl-2-(1- | Tetrahydro Lavandulyl Acetate |
| | methylethyl)-, acetate | |
| 140 | Cyclohexanol, 5-methyl-2-(1- | Iso Pulegol |
| | methylethenyl)-, [1R- | |
| | (1.alpha.,2.beta.,5.alpha.)]- | |
| 141 | Cyclohexanone, 4-(1,1- | Orivone |
| | dimethylpropyl)- | |
| 142 | 2-Undecanone | Methyl Nonyl Ketone |
| 143 | Cyclohexanemethanol, .alpha.,3,3- | Rosamusk |
| | trimethyl-, acetate | |
| 144 | 3-Cyclohexene-1-methanol, 2,4,6- | Isocyclogeraniol |
| | trimethyl- | |
| 145 | 2,6-Octadiene, 1,1-dimethoxy-3,7- | Citral Dimethyl Acetal |
| | dimethyl- | |
| 146 | 1-Decanol | Rhodalione |
| 147 | 2-Cyclohexen-1-one, 3-methyl-5- | Livescone |
| | propyl- | |
| 148 | Phenol, 2-methyl-5-(1-methylethyl)- | Carvacrol |
| 149 | 2-Naphthalenol, decahydro- | Trans Deca Hydro Beta Naphthol |
| 150 | Cyclohexanol, 4-(1,1-dimethylethyl)-, | Tertiary Butyl Cyclohexyl Acetate |
| | acetate | |
| 151 | 9-Decen-1-ol | Rosalva |
| 152 | Phenol, 5-methyl-2-(1-methylethyl)- | Thymol Nf |
| 153 | Cyclohexanol, 5-methyl-2-(1- | Iso Pulegol Acetate |
| | methylethenyl)-, acetate, [1R- | |
| | (1.alpha.,2.beta.,5.alpha.)]- | |
| 154 | Benzene, [(3-methylbutoxy)methyl]- | Iso Amyl Benzyl Ether |
| 155 | 2(3H)-Furanone, 5-hexyldihydro-5- | Lactojasmon |
| | methyl- | |
| 156 | Benzoic acid, butyl ester | Butyl Benzoate |
| 157 | Bicyclo[3.2.1]octan-8-one, 1,5- | Buccoxime |
| | dimethyl-, oxime | |
| 158 | 2-Cyclopenten-1-one, 2-methyl-3-(2- | Iso Jasmone |
| | pentenyl)- | |

In one aspect, suitable Table 2 perfume raw materials include perfume raw materials from number 1 to number 116 and mixtures thereof.

In one aspect, suitable Table 2 perfume raw materials include perfume raw materials from number 1 to number 79 and mixtures thereof.

In one aspect, suitable perfume raw materials having boiling point greater than 250° C. but less than or equal to 280° C. are those materials listed in Table 3 below and such materials are defined as Table 3 perfume raw materials.

TABLE 3

| Number | Registry Name | Trade Name |
|---|---|---|
| 1 | Dodecanenitrile | Clonal |
| 2 | Cyclohexanepropanoic acid, 2-propenyl ester | Allyl Cyclohexane Propionate |
| 3 | 2-Buten-1-one, 1-(2,6,6-trimethyl-2- | Alpha Damascone |
| | cyclohexen-1-yl)- | |
| 4 | 1,4-Cyclohexanedicarboxylic acid, diethyl | Fructalate |
| | ester | |
| 5 | 2(3H)-Furanone, 5-heptyldihydro- | Undecalactone |
| 6 | Naphthalene, 2-methoxy- | Beta Naphthol Methyl Ether |
| 7 | Benzenepropanal, 4-(1,1-dimethylethyl)- | Bourgeonal |
| 8 | 3-Cyclopentene-1-butanol, .beta.,2,2,3- | Brahmanol |
| | tetramethyl- | |
| 9 | 1H-3a,7-Methanoazulen-6-ol, octahydro- | Cedrol |
| | 3,6,8,8-tetramethyl-,[3R- | |
| | (3.alpha.,3a.beta.,6.alpha.,7.beta.,8a.alpha.)]- | |
| 10 | 2-Propen-1-ol, 3-phenyl-, acetate | Cinnamyl Acetate |
| 11 | Benzenepropanal, .alpha.-methyl-4-(1- | Cymal |
| | methylethyl)- | |
| 12 | 2-Buten-1-one, 1-(2,6,6-trimethyl-1- | Damascone Beta |
| | cyclohexen-1-yl)-, (Z)- | |
| 13 | 2-Buten-1-one, 1-(2,6,6-trimethyl-1,3- | Damascenone |
| | cyclohexadien-1-yl)- | |
| 14 | 2-Buten-1-one, 1-(2,6,6-trimethyl-3- | Delta Damascone |
| | cyclohexen-1-yl)- | |
| 15 | Butanoic acid, 1,1-dimethyl-2-phenylethyl | Dimethyl Benzyl Carbinyl |
| | ester | Butyrate |
| 16 | 2-Dodecenal | 2 Dodecene-1-Al |
| 17 | 2H-Pyran-2-one, 6-heptyltetrahydro- | Dodecalactone |
| 18 | Oxiranecarboxylic acid, 3-methyl-3-phenyl-, | Ethyl Methyl Phenyl Glycidate |
| | ethyl ester | |
| 19 | Oxiranecarboxylic acid, 3-phenyl-, ethyl | Ethyl Phenyl Glycidate |
| | ester | |
| 20 | Phenol, 2-methoxy-4-(2-propenyl)- | Eugenol |
| 21 | Benzenepropanal, .beta.-methyl-3-(1- | Florhydral |
| | methylethyl)- | |
| 22 | Benzenepropanal, 2-ethyl-.alpha.,.alpha.- | Floralozone |
| | dimethyl- | |
| 23 | 4,7-Methano-1H-inden-6-ol, 3a,4,5,6,7,7a- | Frutene |
| | hexahydro-, propanoate | |
| 24 | 2,6-Octadienenitrile, 3,7-dimethyl-, (E)- | Geranyl Nitrile |
| 25 | 1,3-Benzodioxole-5-carboxaldehyde | Heliotropin |
| 26 | Ionone | Ionone Ab |
| 27 | 3-Buten-2-one, 4-(2,6,6-trimethyl-2- | Onone Alpha |
| | cyclohexen-1-yl)-, (E)- | |
| 28 | 3-Buten-2-one, 4-(2,6,6-trimethyl-1- | Ionone Beta |
| | cyclohexen-1-yl)- | |
| 29 | 3-Buten-2-one, 4-(2,6,6-trimethyl-1- | Ionone Beta |
| | cyclohexen-1-yl)-, (E)- | |
| 30 | 3-Buten-2-one, 3-methyl-4-(2,6,6-trimethyl- | Ionone Gamma Methyl |
| | 2-cyclohexen-1-yl)- | |
| 31 | 2-Buten-1-one, 1-(2,4,4-trimethyl-2- | Isodamascone N |
| | cyclohexen-1-yl)-, (E)- | |
| 32 | Phenol, 2-methoxy-4-(1-propenyl)- | Iso Eugenol |
| 33 | 2H-Pyran-4-ol, tetrahydro-3-pentyl-, acetate | Jasmal |
| 34 | Bicyclo[3.1.1]hept-2-ene-2-ethanol, 6,6- | Nopyl Acetate |
| | dimethyl-, acetate | |
| 35 | Benzenepropanol, .alpha.,.alpha.-dimethyl-, | Phenyl Ethyl Dimethyl |
| | acetate | Carbinyl Acetate |
| 36 | Propanoic acid, 2-methyl-, 3a,4,5,6,7,7a- | Cyclabute |
| | hexahydro-4,7-methano-1H- | |
| 37 | Benzaldehyde, 4-hydroxy-3-methoxy- | Vanillin |
| 38 | 3-Cyclohexene-1-carboxaldehyde, 1-methyl- | Vernaldehyde |
| | 4-(4-methylpentyl)- | |
| 39 | Benzenemethanol, ar-methoxy-, acetate | Anisyl Acetate |
| 40 | Bicyclo[2.2.1]hept-5-ene-2-carboxylic acid, | Herbanate Ci |
| | 3-(1-methylethyl)-,ethyl ester, (2-endo,3- | |
| | exo)- | |
| 41 | Butanoic acid, 3-methyl-, 2-phenylethyl ester | Beta Phenyl Ethyl Isovalerate |
| 42 | Benzenepropanal, 4-methoxy-.alpha.-methyl- | Canthoxal |
| 43 | Bicyclo[7.2.0]undec-4-ene, 4,11,11- | Caryophyllene Extra |
| | trimethyl-8-methylene-,[1R-(1R*,4E,9S*)]- | |
| 44 | Cyclohexenebutanal, .alpha.,2,2,6- | Cetonal |
| | tetramethyl- | |
| 45 | 2-Propen-1-ol, 3-phenyl- | Cinnamic Alcohol |
| 46 | 6-Octen-1-ol, 3,7-dimethyl-, propanoate | Citronellyl Propionate |
| 47 | Propanoic acid, decyl ester | N-Decyl Propionate |
| 48 | Phenol, 2-methoxy-4-propyl- | Dihydro Eugenol |
| 49 | Cyclohexanol, 1-ethenyl-2-(1-methylpropyl)-, | Dihydro Ambrate |
| | acetate | |
| 50 | 2-Propenoic acid, 3-phenyl-, ethyl ester | Ethyl Cinnamate |
| 51 | Butanoic acid, 3,7-dimethyl-2,6-octadienyl | Geranyl Butyrate |
| | ester, (E)- | |
| 52 | 2-Octanol, 8,8-dimethoxy-2,6-dimethyl- | Hydroxycitronellal Dimethyl |
| | | Acetal |
| 53 | Cyclohexadieneethanol, 4-(1-methylethyl)-, | Iso Bergamate |
| | formate | |
| 54 | Dodecanal | Lauric Aldehyde |
| 55 | Propanoic acid, 2-methyl-, 1-ethenyl-1,5- | Linalyl Iso Butyrate |
| | dimethyl-4-hexenyl ester | |
| 56 | Benzenepropanol, .beta.,.beta.,3-trimethyl- | Majantol |
| 57 | Benzene, 1,2-dimethoxy-4-(2-propenyl)- | Methyl Eugenol |
| 58 | Propanoic acid, 2-methyl-, 2-phenylethyl | Phenyl Ethyl Iso Butyrate |
| | ester | |
| 59 | 2-Propenenitrile, 3-phenyl- | Cinnamalva |
| 60 | Benzene, [2-(1-propoxyethoxy)ethyl]- | Acetal R |
| 61 | 9-Undecenal, 2,6,10-trimethyl- | Adoxal |
| 62 | 2-Naphthalenol, 1,2,3,4,4a,5,6,7-octahydro- | Ambrinol 20t |
| | 2,5,5-trimethyl- | |
| 63 | 2-Naphthalenol, octahydro-2,5,5-trimethyl- | Ambrinol 20t |
| 64 | Ethanol, 2-[(1,7,7- | Arbanol |
| | trimethylbicyclo[2.2.1]hept-2-yl)oxy]-, exo- | |
| 65 | 1H-2-Benzopyran, 3,4,4a,5,8,8a(or | Bigarade Oxide |
| | 3,4,4a,7,8,8a)-hexahydro-3,3,6,7- | |
| 66 | Cyclohexene, 4-(1,5-dimethyl-4- | Bisabolene |
| | hexenylidene)-1-methyl- | |
| 67 | 1H-3a,7-Methanoazulene, octahydro-6- | Cedramber |
| | methoxy-3,6,8,8-tetramethyl-,[3R- | |
| | (3.alpha.,3a.beta.,6.alpha.,7.beta.,8a.alpha.)]- | |
| 68 | Phenol, 4-chloro-3,5-dimethyl- | 4-Chloro 3,5 Xylenol |
| 69 | 2,6-Octadiene, 1,1-diethoxy-3,7-dimethyl- | Citrathal |
| 70 | Acetaldehyde, [(3,7-dimethyl-6- | Citronellyl Oxyacetaldehyde |
| | octenyl)oxy]- | |
| 71 | Benzenepropanenitrile, .alpha.-ethenyl- | Citrowanil B |
| | .alpha.-methyl- | |
| 72 | Cyclohexanol, 2-(1,1-dimethylpropyl)-, | Coniferan |
| | acetate | |
| 73 | 2H-1-Benzopyran-2-one | Coumarin |
| 74 | 1,3-Nonanediol, monoacetate | Diasmol |
| 75 | Benzene, 1,1′-methylenebis- | Diphenyl Methane |
| 76 | Benzene, 1,1′-oxybis- | Diphenyl Oxide |
| 77 | 1,6-Octadiene, 3-(1-ethoxyethoxy)-3,7- | Elinthal |
| | dimethyl- | |
| 78 | Cyclopentanone, 2-heptyl- | Fleuramone |
| 79 | 5,8-Methano-2H-1-benzopyran-2-one, 6- | Florex |
| | ethylideneoctahydro- | |
| 80 | Octanoic acid, 2-acetyl-, ethyl ester | Gelsone |
| 81 | Indeno[1,2-d]-1,3-dioxin, 4,4a,5,9b- | Indoflor Crist. |
| | tetrahydro- | |
| 82 | Benzeneacetic acid, 2-methylpropyl ester | Iso Butyl Phenylacetate |
| 83 | 2,6-Nonadienenitrile, 3,7-dimethyl- | Lemonile |
| 84 | 3-Decanone, 1-hydroxy- | Methyl Lavender Ketone |
| 85 | Undecane, 1,1-dimethoxy-2-methyl- | Methyl Nonyl Acetaldehyde |
| | | Dimethyl Aceta |
| 86 | 1-Propanone, 1-[2-methyl-5-(1-methylethyl)- | Nerone |
| | 2-cyclohexen-1-yl]- | |
| 87 | 5,9-Undecadienal, 2,6,10-trimethyl- | Oncidal |
| 88 | Quinoline, 6-methyl- | Para Methyl Quinoline |
| 89 | Propanoic acid, 2-methyl-, 2-phenoxyethyl | Phenoxy Ethyl Iso Butyrate |
| | ester | |
| 90 | Ethanol, 2-phenoxy-, propanoate | Phenoxy Ethyl Propionate |
| | | Formerly N-225 |
| 91 | 4,7-Methano-1H-indene-2-carboxaldehyde, | Scentenal |
| | octahydro-5-methoxy- | |
| 92 | 9-Undecen-2-one, 6,10-dimethyl- | Tetra Hydro Psuedo Ionone |
| 93 | Benzenemethanol, .alpha.-(trichloromethyl)-, | Trichloromethyl Phenyl |
| | acetate | Carbinyl Acetate |
| 94 | Phenol, 2-methoxy-4-(methoxymethyl)- | Vaniwhite |
| 95 | Bicyclo[2.2.2]oct-5-ene-2-carboxaldehyde, | Maceal |
| | 6-methyl-8-(1-methylethyl)- | |
| 96 | Benzene, [2-(3-methylbutoxy)ethyl]- | Phenyl Ether Isamyl Ether (Aka |
| | | Anther) |
| 97 | 2-Cyclohexene-1-carboxylic acid, 2,3,6,6- | Givescone |
| | tetramethyl-, ethyl ester | |

In one aspect, suitable Table 3 perfume raw materials include perfume raw materials from number 1 to number 58 and mixtures thereof.

In one aspect, suitable Table 3 perfume raw materials include perfume raw materials from number 1 to number 39 and mixtures thereof.

Suitable perfume raw materials and accords may be obtained from one or more of the following companies Firmenich (Geneva, Switzerland), Givaudan (Argenteuil, France), IFF (Hazlet, N.J. U.S.A.), Quest (Mount Olive, N.J. U.S.A), Bedoukian (Danbury, Conn.), Sigma Aldrich (St. Louis, Mo. U.S.A), Millennium Specialty Chemicals (Olympia Fields, Ill. U.S.A), Polarone International (Jersey City, N.J. U.S.A), Fragrance Resources (Keyport, N.J. U.S.A), and Aroma & Flavor Specialties (Danbury, Conn. U.S.A).

Non-limiting examples of suitable perfume delivery systems that may be used in any combination, including mixtures thereof, in the consumer product disclosed herein are:
Polymer Assisted Delivery (PAD): This technology uses polymeric materials to deliver perfume materials. Classical coacervation, water soluble or partly soluble charged polymers, liquid crystals, hot melts, hydrogels, perfumed plastics, microcapsules, nano- and micro-latexes, etc. are examples. PAD includes but is not limited to:
   Matrix Systems: The fragrance is dissolved or dispersed in a polymer matrix or particle. Perfumes can be 1) dispersed into the polymer prior to formulating into the product or 2) added separately from the polymer during or after formulation of the product. Diffusion of perfume is a common trigger that allows or increases the rate of perfume release from a matrix system that has been deposited or applied to the desired surface (situs), although many other triggers are know that can control perfume release. Absorption into or onto polymeric particles also describes this technology. Nano- or micro-particles composed of organic materials (e.g., latexes) are examples. Suitable particles include a wide range of materials including, but not limited to styrene-butadiene copolymers (SBR), acrylonitrile-butadiene copolymers, polychloroprene, acrylic polymers, vinyl acetate polymers, vinyl acetate-ethylene polymers, vinyl chloride polymers and copolymers, polybutadiene and polyisoprene. “Standard” systems refer to those that are “pre-loaded” with the intent of keeping the pre-loaded perfume associated with the polymer until the moment or moments of perfume release. Such polymers can also suppress the neat product odor and provide a bloom and/or longevity benefit depending on the rate of perfume release. One challenge with such systems is to achieve the ideal balance between 1) in-product stability (keeping perfume inside carrier until you need it) and 2) timely release (during use or from dry situs). Achieving such stability is particularly important during in-product storage and product aging. This challenge is particularly apparent for aqueous-based, surfactant-containing products, such as heavy duty liquid laundry detergents. Many “Standard” matrix systems available effectively become “Equilibrium” systems when formulated into aqueous-based products. One can select an “Equilibrium” system or a Reservoir system, which can have acceptable in-product diffusion stability and have available triggers for release (e.g., friction). “Equilibrium” systems are those in which the perfume and polymer can be added separately to the product, and the equilibrium interaction between perfume and polymer leads to a benefit at one or more consumer touch points (versus a free perfume control that has no polymer-assisted delivery technology). The polymer can also be pre-loaded with perfume; however, part or all of the perfume can diffuse during in-product storage reaching an equilibrium that includes having desired perfume raw materials (PRMs) associated with the polymer. The polymer then carries the perfume to the surface, and release is typically via simple perfume diffusion. The use of such equilibrium system polymers has the potential to decrease the neat product odor intensity of the neat product (more so in the case of pre-loaded standard system). Deposition of such polymers can serve to “flatten” the release profile and provide increased longevity. As indicated above, such longevity would be achieved by suppressing the initial intensity and can enable the formulator to use more high impact PRMs to achieve FMOT benefits without causing initial intensity to be too strong or to be distorted. It is important that perfume release occurs within the time frame of the application to impact the desired consumer touch point or touch points. Suitable micro-particles and micro-latexes as well as making same can be found in USPA 2005/0003980 A1. Matrix systems also include hot melt adhesives and perfume plastics. Suitable hot melt adhesives and perfume plastics include those described in USPAs 2003/0109628 A1; 2004/0018950 A1; 2004/0063865 A1; 2004/0059310 A1; 2004/0059018 A1; 2005/0026801 A1; 2005/0106200 A1; 2005/0272878 A1; 2005/0147523 A1; 2006/0029564 A1; 2006/0099168 A1 and U.S. Pat. Nos. 6,498,201 B1 and 6,534,561 B1. In addition, hydrophobically modified polysaccharides can be formulated into the perfumed product to increase perfume deposition and/or modify perfume release. All such matrix systems, including for example polysaccarides and nanolatexes can be combined with other PDTs such as perfume microcapsules.
   Silicones are also examples of polymers that may be used as PDT, and can provide perfume benefits in a manner similar to the polymer-assisted delivery “matrix system”. Such a PDT is referred to as silicone-assisted delivery (SAD). One can pre-load silicones, or use them as an equilibrium system as described for PAD. Suitable silicones as well as making same can be found in WO 2005/102261; USPA 20050124530A1; USPA 20050143282A1; WO 2003/015736. Functionalized silicones may also be used as described in USPA 2006/003913 A1. Examples of silicones include polydimethylsiloxane and polyalkyldimethylsiloxanes. Other examples include those with amine functionality, which can be used to provide benefits associated with amine-assisted delivery (AAD) and/or polymer-assisted delivery (PAD) and/or amine-reaction products (ARP). Other examples include, such as U.S. Pat. No. 4,911,852, USPA 2004/0058845 A1, and USPA 2004/0092425 A1.
   Reservoir Systems: Reservoir systems can be described as a core-shell type technology, or one in which the fragrance is surrounded by a perfume release controlling membrane (protective shell). Microparticles or pressure sensitive capsules are examples of this technology. The possible shell materials vary widely in their stability toward water. Among the most stable are polyoxymethyleneurea (PMU)-based materials, which can hold certain PRMs for even long periods of time in aqueous solution (or product). Such systems include but are not limited to urea-formaldehyde and/or melamine-formaldehyde. Gelatin-based microcapsules can be prepared to dissolve quickly or slowly in water, depending for example on the degree of cross-linking. Many other capsule wall materials are available and vary in the degree of perfume diffusion stability observed. The rate of release of perfume from a capsule, for example, once deposited on a surface is typically in reverse order of in-product stability. As such, urea-formaldehyde and melamine-formaldehyde microcapsules for example, typically require a release mechanism other than, or in addition to, diffusion for release, such as mechanical force (e.g., friction) that serves to break the capsule and increase the rate of perfume (fragrance) release. The use of pre-loaded microcapsules requires the proper ratio of in-product stability and in-use and/or on-surface (on-situs) release, as well as proper selection of PRMs. Microcapsules that are based on urea-formaldehyde and/or melamine-formaldehyde are relatively stable, especially in near neutral aqueous-based solutions. These materials can require a friction trigger which may not be suitable to all product applications. Other microcapsule materials (e.g., gelatin) can be unstable in water and can even provide reduced benefit (versus free perfume control) when in-product aged. Perfume microcapsules (PMC) may include those described in the following references: US Patent Applications: 2003/0125222 A1; 2003/215417 A1; 2003/216488 A1; 2003/158344 A1; 2003/165692 A1; 2004/071742 A1; 2004/071746 A1; 2004/072719 A1; 2004/072720 A1; 2006/0039934A1; 2003/203829 A1; 2003/195133 A1; 2004/087477 Al; 2004/0106536 A1; and U.S. Pat. Nos. 6,645,479 B1; 6,200,949 B1; 4,882,220; 4,917,920; 4,514,461; and 4,234,627, and US RE 32713. In one aspect, said perfume microcapsule comprises, based on total perfume microcapsule weight, from about 50% to about 95%, from about 60% to about 90%, from about 75% to about 85% perfume, at least 50%, 75%, 85% or even 100% said perfume being a perfume raw material having a ClogP great than or equal to 1 and a boiling point less than or equal to 350° C.; a ClogP great than or equal to 1.5 and a boiling point less than or equal to 300° C.; or even a ClogP great than or equal to 2 and a boiling point less than or equal to 280° C. For purposes of the present invention, and unless indicated otherwise, the terms “perfume nanocapsule” and “microcapsule” is within the scope of the term “perfume microcapsule.”
Monomer-Assisted Delivery (MAD): Non-polymer materials or molecules can also serve to improve the delivery of perfume. Without wishing to be bound by theory, perfume can non-covalently interact with organic materials, resulting in altered deposition and/or release. Non-limiting examples of such organic materials include but are not limited to hydrophobic materials such as organic oils, mineral oils, petrolatum, fatty acids or esters, sugars, surfactants, and even other perfume raw material (perfume oils). Useful non-polymeric materials or molecules include those with a CLogP greater than about 2.
Amine Assisted Delivery (AAD): The amine-assisted delivery technology approach utilizes materials that contain an amine group to increase perfume deposition or modify perfume release during product use. There is no requirement in this approach to pre-complex or pre-react the perfume raw material(s) and amine prior to addition to the product. Materials that can contain an amine group and be suitable for use herein may be non-aromatic, for example, polyalkylimine, such as polyethyleneimine (PEI), or polyvinylamine (PVAm), or aromatic, for example, anthranilates. Such materials may also be polymeric or non-polymeric. In one aspect, such materials contain at least one primary amine. This technology will allow increased longevity and controlled release also of low ODT perfume notes (e.g., aldehydes, ketones, enones) via amine functionality, and delivery of other PRMs, without being bound by theory, via polymer-assisted delivery for polymeric amines. Without technology, volatile top notes can be lost too quickly, leaving a higher ratio of middle and base notes to top notes. The use of a polymeric amine allows higher levels of top notes and other PRMS to be used to obtain freshness longevity without causing neat product odor to be more intense than desired, or allows top notes and other PRMs to be used more efficiently. Suitable AAD systems as well as methods of making same can be found in US Patent Applications 2005/0003980 A1; 2003/0199422 A1; 2003/0036489 A1 and U.S. Pat. No. 6,103,678.
Cyclodextrins (CD): This technology approach uses cyclodextrin to improve the delivery of perfume. Typically a perfume and cyclodextrin (CD) complex is formed. Such complexes can be preformed, formed in-situ, or even formed upon the situs. Without wishing to be bound by theory, loss of water can serve to shift the equilibrium toward the CD-Perfume complex, especially if other adjunct ingredients (e.g., surfactant) are not present at high concentration to compete with the perfume for the cyclodextrin cavity. A bloom benefit can be achieved if water exposure occurs at a later time point. In addition, cyclodextrin allows the perfume formulator increased flexibility in selection of PRMs. Cyclodextrin can be preloaded with perfume or added separately from perfume to obtain the desired perfume stability, deposition or release benefit. Suitable CDs as well as methods of making same can be found in USPA 2005/0003980 A1 and U.S. Pat. Nos. 5,552,378; 3,812,011; 4,317,881; 4,418,144; and 4,378,923.
Starch Encapsulated Accords (SEA): The use of a starch encapsulated accord (SEA) technology allows one to modify the properties of the perfume, for example, by converting a liquid perfume into a solid by adding ingredients such as starch. The benefit includes increased perfume retention during product storage, especially under non-aqueous conditions. Upon exposure to moisture, a perfume bloom can be triggered. Benefits at other moments of truth can also be achieved because the starch allows the product formulator to select PRMs or PRM concentrations that normally cannot be used without the presence of SEA. Another technology example includes the use of other organic and inorganic materials, such as silica to convert perfume from liquid to solid. Suitable SEAs as well as methods of making same can be found in USPA 2005/0003980 A1 and U.S. Pat. No. 6,458,754 B1.
Inorganic Carriers (IC): This technology relates to the use of porous zeolites or other inorganic materials to deliver perfumes. Perfume-loaded zeolite can be used with or without adjunct ingredients used for example to coat the perfume-loaded zeolite (PLZ) to change its perfume release properties during product storage or during use or from the dry situs. Suitable zeolite and inorganic carriers as well as methods of making same can be found in USPA 2005/0003980 A1 and U.S. Pat. Nos. 5,858,959, 6,245,732 B1 and 6,048,830. Another example of a suitable inorganic carrier includes inorganic tubules, where the perfume or other active material is contained within the lumen of the nano- or micro-tubules. In one aspect, the perfume-loaded inorganic tubule (or Perfume-Loaded Tubule or PLT) is a mineral nano- or micro-tubule, such as halloysite or mixtures of halloysite with other inorganic materials, including other clays. The PLT technology can also comprise additional ingredients on the inside and/or outside of the tubule for the purpose of improving in-product diffusion stability, deposition on the desired situs or for controlling the release rate of the loaded perfume. Monomeric and/or polymeric materials, including starch encapsulation, can be used to coat, plug, cap, or otherwise encapsulate the PLT. Suitable PLTs as well as methods of making same can be found in U.S. Pat. No. 5,651,976.
Pro-Perfumes (PP): This technology refers to perfume technologies that result from the reaction of perfume materials with other substrates or chemicals to form materials that have a covalent bond between PRM and carrier. The PRM is converted into a new material called a pro-PRM (i.e., pro-perfume), which then releases the original PRM upon exposure to a trigger such as water or light. Pro-perfumes can provide enhanced perfume delivery properties such as increased deposition, longevity, stability, retention, and the like. Pro-perfumes include those that are monomeric or polymeric, and can be pre-formed or can be formed in-situ under equilibrium conditions, such as those present during in-product storage or on the wet or dry situs. Nonlimiting examples of pro-perfumes include Michael adducts (e.g., beta-amino ketones), aromatic or non-aromatic imines (Schiff bases), oxazolidines, beta-keto esters, and orthoesters, The typical trigger for perfume release is exposure to water; although other triggers may include enzymes, heat, light, pH change, autoxidation, shift of equilibrium, change in concentration and others. For aqueous-based products, light triggered pro-perfumes are particularly suited. Such photo-pro-perfumes (PPPs) include but are not limited to those that release coumarin derivatives and pro-perfumes upon being triggered. The released pro-perfume can release PRM(s) by means of any of the above mentioned triggers. Suitable pro-perfumes and methods of making same can be found in U.S. Pat. Nos. 7,018,978 B2; 6,987,084 B2; 6,956,013 B2; 6,861,402 B1; 6,544,945 B1; 6,093,691; 6,277,796 B1; 6,165,953; 6,316,397 B1; 6,437,150 B1; 6,479,682 B1; 6,096,918; 6,218,355 B1; 6,133,228; 6,147,037; and 5,958,870.
Amine Reaction Products (ARP): One can also use “reactive” polymeric amines in which the amine functionality is pre-reacted with PRMs to form an amine reaction product (ARP). Typically the reactive amines are primary and/or secondary amines, and can be part of a polymer or a monomer. Such ARPs can also be mixed with additional PRMs to provide benefits of polymer-assisted delivery and/or amine-assisted delivery. Nonlimiting examples of monomeric amines include hydroxyl amines and aromatic amines such as anthranilates. The ARPs can be premixed with perfume or added separately in leave-on or rinse-off applications. The benefit can include improved delivery of perfume as well as controlled release. Suitable ARPs as well as methods of making same can be found in USPA 2005/0003980 A1 and U.S. Pat. No. 6,413,920 B1.

Suitable laundry care ingredients include, but are not limited to, those materials described in the present specification as useful aspects of the present invention, including adjunct materials as described in the present specification.

### Liquid, Laundry Detergent Compositions

In one aspect, the laundry care compositions disclosed herein, may take the form of liquid, laundry detergent compositions. In one aspect, such compositions may be a heavy duty liquid composition. Such compositions may comprise a sufficient amount of a surfactant to provide the desired level of one or more cleaning properties, typically by weight of the total composition, from about 5% to about 90%, from about 5% to about 70% or even from about 5% to about 40%. The liquid detergent compositions comprise an aqueous, non-surface active liquid carrier. Generally, the amount of the aqueous, non-surface active liquid carrier employed in the compositions herein will be effective to solubilize, suspend or disperse the composition components. For example, the compositions may comprise, by weight, from about 5% to about 90%, from about 10% to about 70%, or even from about 20% to about 70% of an aqueous, non-surface active liquid carrier.

The most cost effective type of aqueous, non-surface active liquid carrier may be water. Accordingly, the aqueous, non-surface active liquid carrier component may be generally mostly, if not completely, water. While other types of water-miscible liquids, such alkanols, diols, other polyols, ethers, amines, and the like, have been conventionally been added to liquid detergent compositions as co-solvents or stabilizers, for purposes of the present invention, the utilization of such water-miscible liquids may be minimized to hold down composition cost. Accordingly, the aqueous liquid carrier component of the liquid detergent products herein will generally comprise water present in concentrations ranging from about 5% to about 90%, or even from about 20% to about 70%, by weight of the composition.

The liquid detergent compositions herein may take the form of an aqueous solution or uniform dispersion or suspension of surfactant, and certain optional other ingredients, some of which may normally be in solid form, that have been combined with the normally liquid components of the composition, such as the liquid alcohol ethoxylate nonionic, the aqueous liquid carrier, and any other normally liquid optional ingredients. Such a solution, dispersion or suspension will be acceptably phase stable and will typically have a viscosity which ranges from about 100 to 600 cps, or even from about 150 to 400 cps. For purposes of this invention, viscosity is measured with a Brookfield LVDV-II+ viscometer apparatus using a #21 spindle.

Suitable surfactants may be anionic, nonionic, cationic, zwitterionic and/or amphoteric surfactants. In one aspect, the detergent composition comprises anionic surfactant, nonionic surfactant, or mixtures thereof.

Suitable anionic surfactants may be any of the conventional anionic surfactant types typically used in liquid detergent products. Such surfactants include the alkyl benzene sulfonic acids and their salts as well as alkoxylated or non-alkoxylated alkyl sulfate materials. Exemplary anionic surfactants are the alkali metal salts of C₁₀-C₁₆ alkyl benzene sulfonic acids, including C₁₁-C₁₄ alkyl benzene sulfonic acids. In one aspect, the alkyl group is linear. Such linear alkyl benzene sulfonates are known as “LAS”. Such surfactants and their preparation are described for example in U.S. Pat. Nos. 2,220,099 and 2,477,383. In one aspect, sodium and potassium linear straight chain alkylbenzene sulfonates in which the average number of carbon atoms in the alkyl group is from about 11 to 14 may be used. Sodium C₁₁-C₁₄, e.g., C₁₂, LAS is a specific example of such surfactants.

Another exemplary type of anionic surfactant comprises ethoxylated alkyl sulfate surfactants. Such materials, also known as alkyl ether sulfates or alkyl polyethoxylate sulfates, are those which correspond to the formula: R′—O—(C₂H4O)n-SO₃M wherein R′ is a C₈-C₂₀ alkyl group, n is from about 1 to 20, and M is a salt-forming cation. In a specific embodiment, R′ is C₁₀-C₁₈ alkyl, n is from about 1 to 15, and M is sodium, potassium, ammonium, alkylammonium, or alkanolammonium. In more specific embodiments, R′ is a C₁₂-C₁₆, n is from about 1 to 6 and M is sodium.

The alkyl ether sulfates will generally be used in the form of mixtures comprising varying R′ chain lengths and varying degrees of ethoxylation. Frequently such mixtures will inevitably also contain some non-ethoxylated alkyl sulfate materials, i.e., surfactants of the above ethoxylated alkyl sulfate formula wherein n=0. Non-ethoxylated alkyl sulfates may also be added separately to the compositions of this invention and used as or in any anionic surfactant component which may be present. Specific examples of non-alkoyxylated, e.g., non-ethoxylated, alkyl ether sulfate surfactants are those produced by the sulfation of higher C₈-C₂₀ fatty alcohols. Conventional primary alkyl sulfate surfactants have the general formula: ROSO₃-M+ wherein R is typically a linear C₈-C₂₀ hydrocarbyl group, which may be straight chain or branched chain, and M is a water-solubilizing cation. In specific embodiments, R is a C₁₀-C₁₅ alkyl, and M is alkali metal, more specifically R is C₁₂-C₁₄ and M is sodium.

Specific, nonlimiting examples of anionic surfactants useful herein include: a) C₁₁-C₁₈ alkyl benzene sulfonates (LAS); b) C₁₀-C₂₀ primary, branched-chain and random alkyl sulfates (AS); c) C₁₀-C₁₈ secondary (2,3) alkyl sulfates having formulae (I) and (II):

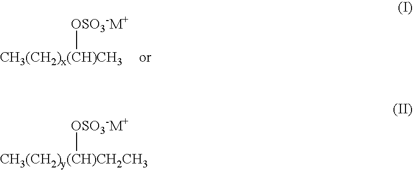

wherein M in Formulae (I) and (II) is hydrogen or a cation which provides charge neutrality, and all M units, whether associated with a surfactant or adjunct ingredient, can either be a hydrogen atom or a cation depending upon the form isolated by the artisan or the relative pH of the system wherein the compound is used, with non-limiting examples of cations including sodium, potassium, ammonium, and mixtures thereof, and x is an integer of at least about 7, or even at least about 9, and y is an integer of at least 8, or even at least about 9; d) C₁₀-C₁₈ alkyl alkoxy sulfates (AExS) wherein x may be from 1-30; e) C₁₀-C₁₈ alkyl alkoxy carboxylates or even comprising 1-5 ethoxy units; f) mid-chain branched alkyl sulfates as discussed in U.S. Pat. No. 6,020,303 and U.S. Pat. No. 6,060,443; g) mid-chain branched alkyl alkoxy sulfates as discussed in U.S. Pat. No. 6,008,181 and U.S. Pat. No. 6,020,303; h) modified alkylbenzene sulfonate (MLAS) as discussed in WO 99/05243, WO 99/05242, WO 99/05244, WO 99/05082, WO 99/05084, WO 99/05241, WO 99/07656, WO 00/23549, and WO 00/23548.; i) methyl ester sulfonate (MES); and j) alpha-olefin sulfonate (AOS).

Suitable nonionic surfactants useful herein can comprise any of the conventional nonionic surfactant types typically used in liquid detergent products. These include alkoxylated fatty alcohols and amine oxide surfactants. In one aspect, those nonionic surfactants which are normally liquid are used.

Suitable nonionic surfactants for use herein include the alcohol alkoxylate nonionic surfactants. Alcohol alkoxylates are materials which correspond to the general formula: R1(CmH₂mO)nOH wherein R1 is a C₈-C₁₆ alkyl group, m is from 2 to 4, and n ranges from about 2 to 12. In one aspect, R1 is an alkyl group, which may be primary or secondary, that contains from about 9 to 15 carbon atoms, or even from about 10 to 14 carbon atoms. In one embodiment, the alkoxylated fatty alcohols will also be ethoxylated materials that contain from about 2 to 12 ethylene oxide moieties per molecule, or even from about 3 to 10 ethylene oxide moieties per molecule.

The alkoxylated fatty alcohol materials useful in the liquid detergent compositions herein will frequently have a hydrophilic-lipophilic balance (HLB) which ranges from about 3 to 17. In one aspect, the HLB of this material will range from about 6 to 15, or even from about 8 to 15. Alkoxylated fatty alcohol nonionic surfactants have been marketed under the tradename Neodol® by the Shell Chemical Company.

Another suitable type of nonionic surfactant useful herein comprises the amine oxide surfactants. Amine oxides are materials which are often referred to in the art as “semi-polar” nonionics. Amine oxides have the formula: R(EO)x(PO)y(BO)zN(O)(CH2R′)₂.qH₂O. In this formula, R is a relatively long-chain hydrocarbyl moiety which can be saturated or unsaturated, linear or branched, and can contain from 8 to 20, from 10 to 16 carbon atoms, or even C₁₂-C₁₆ primary alkyl. R′ is a short-chain moiety, that, in one aspect, is selected from hydrogen, methyl and —CH₂OH. When x+y+z is different from 0, EO is ethyleneoxy, PO is propyleneneoxy and BO is butyleneoxy. Amine oxide surfactants are illustrated by C₁₂-C₁₄ alkyldimethyl amine oxide. Non-limiting examples of nonionic surfactants include: a) C₁₂-C₁₈ alkyl ethoxylates, such as, NEODOL® nonionic surfactants from Shell; b) C₆-C₁₂ alkyl phenol alkoxylates wherein the alkoxylate units are a mixture of ethyleneoxy and propyleneoxy units; c) C₁₂-C₁₈ alcohol and C₆-C₁₂ alkyl phenol condensates with ethylene oxide/propylene oxide block polymers such as Pluronic® from BASF; d) C₁₄-C₂₂ mid-chain branched alcohols, BA, as discussed in U.S. Pat. No. 6,150,322; e) C₁₄-C₂₂ mid-chain branched alkyl alkoxylates, BAEx, wherein x 1-30, as discussed in U.S. Pat. No. 6,153,577, U.S. Pat. No. 6,020,303 and U.S. Pat. No. 6,093,856; f) Alkylpolysaccharides as discussed in U.S. Pat. No. 4,565,647 Llenado, issued Jan. 26, 1986; specifically alkylpolyglycosides as discussed in U.S. Pat. No. 4,483,780 and U.S. Pat. No. 4,483,779; g) Polyhydroxy fatty acid amides as discussed in U.S. Pat. No. 5,332,528, WO 92/06162, WO 93/19146, WO 93/19038, and WO 94/09099; and h) ether capped poly(oxyalkylated) alcohol surfactants as discussed in U.S. Pat. No. 6,482,994 and WO 01/42408.

In the laundry detergent compositions herein, the detersive surfactant component may comprise combinations of anionic and nonionic surfactant materials. When this is the case, the weight ratio of anionic to nonionic will typically range from 10:90 to 90:10, more typically from 30:70 to 70:30.

Cationic surfactants are well known in the art and non-limiting examples of these include quaternary ammonium surfactants, which can have up to 26 carbon atoms. Additional examples include a) alkoxylate quaternary ammonium (AQA) surfactants as discussed in U.S. Pat. No. 6,136,769; b) dimethyl hydroxyethyl quaternary ammonium as discussed in U.S. Pat. No. 6,004,922; c) polyamine cationic surfactants as discussed in WO 98/35002, WO 98/35003, WO 98/35004, WO 98/35005, and WO 98/35006; d) cationic ester surfactants as discussed in U.S. Pat. Nos. 4,228,042, 4,239,660 4,260,529 and U.S. Pat. No. 6,022,844; and e) amino surfactants as discussed in U.S. Pat. No. 6,221,825 and WO 00/47708, specifically amido propyldimethyl amine (APA).

Non-limiting examples of zwitterionic surfactants include: derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 to Laughlin et al., issued Dec. 30, 1975, at column 19, line 38 through column 22, line 48, for examples of zwitterionic surfactants; betaine, including alkyl dimethyl betaine and cocodimethyl amidopropyl betaine, C₈ to C₁₈ (in one aspect C₁₂ to C₁₈) amine oxides and sulfo and hydroxy betaines, such as N-alkyl-N,N-dimethylammino-1-propane sulfonate where the alkyl group can be C₈ to C₁₈, or even C₁₀ to C₁₄.

Non-limiting examples of ampholytic surfactants include: aliphatic derivatives of secondary or tertiary amines, or aliphatic derivatives of heterocyclic secondary and tertiary amines in which the aliphatic radical can be straight- or branched-chain. One of the aliphatic substituents contains at least about 8 carbon atoms, typically from about 8 to about 18 carbon atoms, and at least one contains an anionic water-solubilizing group, e.g. carboxy, sulfonate, sulfate. See U.S. Pat. No. 3,929,678 to Laughlin et al., issued Dec. 30, 1975, at column 19, lines 18-35, for examples of ampholytic surfactants.

### Granular Laundry Detergent Compositions

In one aspect, the laundry care compositions disclosed herein, may take the form of granular, laundry detergent compositions. Granular detergent compositions of the present invention may include any number of conventional detergent ingredients. For example, the surfactant system of the detergent composition may include anionic, nonionic, zwitterionic, ampholytic and cationic classes and compatible mixtures thereof. Detergent surfactants for granular compositions are described in U.S. Pat. No. 3,664,961, Norris, issued May 23, 1972, and in U.S. Pat. No. 3,919,678, Laughlin et al., issued Dec. 30, 1975. Cationic surfactants include those described in U.S. Pat. No. 4,222,905, Cockrell, issued Sep. 16, 1980, and in U.S. Pat. No. 4,239,659, Murphy, issued Dec. 16, 1980.

Nonlimiting examples of surfactant systems include the conventional C₁₁-C₁₈ alkyl benzene sulfonates (“LAS”) and primary, branched-chain and random C₁₀-C₂₀ alkyl sulfates (“AS”), the C₁₀-C₁₈ secondary (2,3) alkyl sulfates of the formula CH₃(CH₂)x(CHOSO₃—M+) CH₃ and CH₃ (CH₂)y(CHOSO₃M+) CH₂CH₃ where x and (y+1) are integers of at least about 7, or even at least about 9, and M is a water-solubilizing cation, especially sodium, unsaturated sulfates such as oleyl sulfate, the C₁₀-C₁₈ alkyl alkoxy sulfates (“AExS”; especially EO 1-7 ethoxy sulfates), C₁₀-C₁₈ alkyl alkoxy carboxylates (especially the EO 1-5 ethoxycarboxylates), the C₁₀-C₁₈ glycerol ethers, the C₁₀-C₁₈ alkyl polyglycosides and their corresponding sulfated polyglycosides, and C₁₂-C₁₈ alpha-sulfonated fatty acid esters. If desired, the conventional nonionic and amphoteric surfactants such as the C₁₂-C₁₈ alkyl ethoxylates (“AE”) including the so-called narrow peaked alkyl ethoxylates and C₆-C₁₂ alkyl phenol alkoxylates (especially ethoxylates and mixed ethoxy/propoxy), C₁₂-C₁₈ betaines and sulfobetaines (“sultaines”), C₁₀-C₁₈ amine oxides, and the like, can also be included in the surfactant system. The C₁₀-C₁₈ N-alkyl polyhydroxy fatty acid amides can also be used. Other sugar-derived surfactants include the N-alkoxy polyhydroxy fatty acid amides, such as C₁₀-C₁₈ N-(3-methoxypropyl) glucamide. The N-propyl through N-hexyl C₁₂-C₁₈ glucamides can be used for low sudsing. C₁₀-C₂₀ conventional soaps may also be used. If high sudsing is desired, the branched-chain C₁₀-C₁₆ soaps may be used. Mixtures of anionic and nonionic surfactants are especially useful. Other conventional useful surfactants are listed in standard texts.

The detergent composition can, and, in one or more aspects does, include a detergent builder. Builders are generally selected from the various water-soluble, alkali metal, ammonium or substituted ammonium phosphates, polyphosphates, phosphonates, polyphosphonates, carbonates, silicates, borates, polyhydroxy sulfonates, polyacetates, carboxylates, and polycarboxylates. In one aspect, suitable builders include alkali metals, especially sodium, salts of the above. In one aspect, phosphates, carbonates, silicates, C₁₀-C₁₈ fatty acids, polycarboxylates, and mixtures thereof may be used. In another aspect, sodium tripolyphosphate, tetrasodium pyrophosphate, citrate, tartrate mono and di succinates, sodium silicate, and mixtures thereof may be used.

Specific examples of inorganic phosphate builders are sodium and potassium tripolyphosphate, pyrophosphate, polymeric metaphosphate having a degree of polymerization of from about 6 to 21, and orthophosphates. Examples of polyphosphonate builders are the sodium and potassium salts of ethylene diphosphonic acid, the sodium and potassium salts of ethane 1-hydroxy-1, 1-diphosphonic acid and the sodium and potassium salts of ethane, 1,1,2-triphosphonic acid. Other phosphorus builder compounds are disclosed in U.S. Pat. Nos. 3,159,581; 3,213,030; 3,422,021; 3,422,137; 3,400,176 and 3,400,148. Examples of nonphosphorus, inorganic builders are sodium and potassium carbonate, bicarbonate, sesquicarbonate, tetraborate decahydrate, and silicates having a weight ratio of SiO₂ to alkali metal oxide of from about 0.5 to about 4.0, or even from about 1.0 to about 2.4. Water-soluble, nonphosphorus organic builders useful herein include the various alkali metal, ammonium and substituted ammonium polyacetates, carboxylates, polycarboxylates and polyhydroxy sulfonates. Examples of polyacetate and polycarboxylate builders are the sodium, potassium, lithium, ammonium and substituted ammonium salts of ethylene diamine tetraacetic acid, nitrilotriacetic acid, oxydisuccinic acid, mellitic acid, benzene polycarboxylic acids, and citric acid.

Polymeric polycarboxylate builders are set forth in U.S. Pat. No. 3,308,067, Diehl, issued Mar. 7, 1967. Such materials include the water-soluble salts of homo- and copolymers of aliphatic carboxylic acids such as maleic acid, itaconic acid, mesaconic acid, fumaric acid, aconitic acid, citraconic acid and methylenemalonic acid. Some of these materials are useful as the water-soluble anionic polymer as hereinafter described, but only if in intimate admixture with the nonsoap anionic surfactant. Other suitable polycarboxylates for use herein are the polyacetal carboxylates described in U.S. Pat. No. 4,144,226, issued Mar. 13, 1979, to Crutchfield et al., and U.S. Pat. No. 4,246,495, issued Mar. 27, 1979, to Crutchfield et al.

Water-soluble silicate solids represented by the formula SiO₂.M₂O, M being an alkali metal, and having a SiO₂:M₂O weight ratio of from about 0.5 to about 4.0, are useful salts in the detergent granules of the invention at levels of from about 2% to about 15% on an anhydrous weight basis. Anhydrous or hydrated particulate silicate can be utilized, as well.

Any number of additional ingredients can also be included as components in the granular detergent composition. These include other detergency builders, bleaches, bleach activators, suds boosters or suds suppressors, anti tarnish and anti-corrosion agents, soil suspending agents, soil release agents, germicides, pH adjusting agents, nonbuilder alkalinity sources, chelating agents, smectite clays, enzymes, enzyme stabilizing agents and perfumes. See U.S. Pat. No. 3,936,537, issued Feb. 3, 1976, to Baskerville, Jr. et al.

Bleaching agents and activators are described in U.S. Pat. No. 4,412,934, Chung et al., issued Nov. 1, 1983, and in U.S. Pat. No. 4,483,781, Hartman, issued Nov. 20, 1984. Chelating agents are also described in U.S. Pat. No. 4,663,071, Bush et al., from Column 17, line 54 through Column 18, line 68. Suds modifiers are also optional ingredients and are described in U.S. Pat. No. 3,933,672, issued Jan. 20, 1976, to Bartoletta et al., and U.S. Pat. No. 4,136,045, issued Jan. 23, 1979, to Gault et al. Suitable smectite clays for use herein are described in U.S. Pat. No. 4,762,645, Tucker et al., issued Aug. 9, 1988, Column 6, line 3 through Column 7, line 24. Suitable additional detergency builders for use herein are enumerated in the Baskerville patent, Column 13, line 54 through Column 16, line 16, and in U.S. Pat. No. 4,663,071, Bush et al., issued May 5, 1987.

### Rinse Added Fabric Conditioning Compositions

In one aspect, the laundry care compositions disclosed herein, may take the form of rinse added fabric conditioning compositions. Such compositions comprise a fabric softening active. In another embodiment, the compositions are rinse added fabric conditioning compositions. Examples of typical rinse added conditioning composition can be found in U.S. Provisional Patent Application Ser. No. 60/687582 filed on Oct. 8, 2004.

In one embodiment of the invention, the fabric softening active (hereinafter “FSA”) is a quaternary ammonium compound suitable for softening fabric in a rinse step. In one embodiment, the FSA is formed from a reaction product of a fatty acid and an aminoalcohol obtaining mixtures of mono-, di-, and, in one embodiment, triester compounds. In another embodiment, the FSA comprises one or more softener quaternary ammonium compounds such, but not limited to, as a monoalkyquaternary ammonium compound, a diamido quaternary compound and a diester quaternary ammonium compound, or a combination thereof.

In one aspect of the invention, the FSA comprises a diester quaternary ammonium (hereinafter “DQA”) compound composition. In certain embodiments of the present invention, the DQA compounds compositions also encompasses a description of diamido FSAs and FSAs with mixed amido and ester linkages as well as the aforementioned diester linkages, all herein referred to as DQA.

A first type of DQA (“DQA (1)”) suitable as a FSA in the present CFSC includes a compound comprising the formula:

{R4-m-N+-[(CH2)n-Y—R1]m}X—

wherein each R substituent is either hydrogen, a short chain C₁-C₆, or even C₁-C₃ alkyl or hydroxyalkyl group, e.g., methyl, ethyl, propyl, hydroxyethyl, and the like, poly (C₂-C₃ [symbol] alkoxy), polyethoxy, group, benzyl, or mixtures thereof; each m is 2 or 3; each n is from 1 to about 4, or 2; each Y is —O—(O)C—, —C(O)—O—, —NR—C(O)—, or —C(O)—NR— and it is acceptable for each Y to be the same or different; the sum of carbons in each R1, plus one when Y is —O—(O)C— or —NR—C(O)—, is C₁₂-C₂₂, or C₁₄-C₂₀, with each R1 being a hydrocarbyl, or substituted hydrocarbyl group; it is acceptable for R1 to be unsaturated or saturated and branched or linear and in one aspect it is linear; it is acceptable for each R1 to be the same or different and in one aspect these are the same; and X— can be any softener-compatible anion, in one aspect, chloride, bromide, methylsulfate, ethylsulfate, sulfate, phosphate, and nitrate, or chloride or methyl sulfate. Suitable DQA compounds can be made by reacting alkanolamines such as MDEA (methyldiethanolamine) and TEA (triethanolamine) with fatty acids. Some materials that typically result from such reactions include N,N-di(acyl-oxyethyl)-N,N-dimethylammonium chloride or N,N-di(acyl-oxyethyl)-N,N-methylhydroxyethylammonium methylsulfate wherein the acyl group is derived from animal fats, unsaturated, and polyunsaturated, fatty acids, e.g., tallow, hardended tallow, oleic acid, and/or partially hydrogenated fatty acids, derived from vegetable oils and/or partially hydrogenated vegetable oils, such as, canola oil, safflower oil, peanut oil, sunflower oil, corn oil, soybean oil, tall oil, rice bran oil, palm oil, etc. Non-limiting examples of suitable fatty acids are listed in U.S. Pat. No. 5,759,990 at column 4, lines 45-66. In one embodiment the FSA comprises other actives in addition to DQA (1) or DQA. In yet another embodiment, the FSA comprises only DQA (1) or DQA and is free or essentially free of any other quaternary ammonium compounds or other actives. In yet another embodiment, the FSA comprises the precursor amine that is used to produce the DQA.

In another aspect of the invention, the FSA comprises a compound, identified as DTTMAC comprising the formula:

[R4-m-N(+)-R1m]A-

wherein each m is 2 or 3, each R1 is a C₆-C₂₂, or C₁₄-C₂₀, but no more than one being less than about C₁₂ and then the other is at least about 16, hydrocarbyl, or substituted hydrocarbyl substituent, such as C10-C₂₀ alkyl or alkenyl (unsaturated alkyl, including polyunsaturated alkyl, also referred to sometimes as “alkylene”), in one aspect, C₁₂-C₁₈ alkyl or alkenyl, and branch or unbranched. In one embodiment, the Iodine Value (IV) of the FSA is from about 1 to 70; each R is H or a short chain C₁-C₆, such as C₁-C₃ alkyl or hydroxyalkyl group, e.g., methyl, ethyl, propyl, hydroxyethyl, and the like, benzyl, or (R2 O)₂-4H where each R2 is a C₁-C₆ alkylene group; and A- is a softener compatible anion, in one aspect, chloride, bromide, methylsulfate, ethylsulfate, sulfate, phosphate, or nitrate; in another aspect chloride or methyl sulfate. Examples of these FSAs include dialkydimethylammonium salts and dialkylenedimethylammonium salts such as ditallowdimethylammonium and ditallowdimethylammonium methylsulfate. Examples of commercially available dialkylenedimethylammonium salts usable in the present invention are di-hydrogenated tallow dimethyl ammonium chloride and ditallowdimethyl ammonium chloride available from Degussa under the trade names Adogen® 442 and Adogen® 470 respectively. In one embodiment the FSA comprises other actives in addition to DTTMAC. In yet another embodiment, the FSA comprises only compounds of the DTTMAC and is free or essentially free of any other quaternary ammonium compounds or other actives.

In one embodiment, the FSA comprises an FSA described in U.S. Pat. Pub. No. 2004/0204337 A1, published Oct. 14, 2004, to Corona et al., from paragraphs 30-79.

In another embodiment, the FSA is one described in U.S. Pat. Pub. No. 2004/0229769 A1, published Nov. 18, 2005, to Smith et al., on paragraphs 26-31; or U.S. Pat. No. 6,494,920, at column 1, line 51 et seq. detailing an “esterquat” or a quaternized fatty acid triethanolamine ester salt.

In one embodiment, the FSA is chosen from at least one of the following: ditallowoyloxyethyl dimethyl ammonium chloride, dihydrogenated-tallowoyloxyethyl dimethyl ammonium chloride, ditallow dimethyl ammonium chloride, ditallowoyloxyethyl dimethyl ammonium methyl sulfate, dihydrogenated-tallowoyloxyethyl dimethyl ammonium chloride, dihydrogenated-tallowoyloxyethyl dimethyl ammonium chloride, or combinations thereof.

In one embodiment, the FSA may also include amide containing compound compositions. Examples of diamide comprising compounds may include but not limited to methyl-bis(tallowamidoethyl)-2-hydroxyethylammonium methyl sulfate (available from Degussa under the trade names Varisoft 110 and Varisoft 222). An example of an amide-ester containing compound is N-[3-(stearoylamino)propyl]-N-[2-(stearoyloxy)ethoxy)ethyl)]-N-methylamine.

Another specific embodiment of the invention provides for a rinse added fabric care composition further comprising a cationic starch. Cationic starches are disclosed in US 2004/0204337 A1. In one embodiment, the fabric care composition comprises from about 0.1% to about 7% of cationic starch by weight of the laundry care composition. In one embodiment, the cationic starch is HCP401 from National Starch.

### Adjunct Materials

While not essential for the purposes of the present invention, the non-limiting list of adjuncts illustrated hereinafter are suitable for use in the consumer products disclosed herein and may be desirably incorporated in certain embodiments of such products, for example to assist or enhance performance, for treatment of the substrate to be cleaned, or to modify the aesthetics of the composition as is the case with perfumes, colorants, dyes or the like. It is understood that when a previous component, other than a perfume raw material or a perfume delivery system, is listed for an embodiment such adjuncts are in addition to the components that were previously listed for any particular embodiment. The total amount of such adjuncts may range from about 0.1% to about 50%, or even from about 1% to about 30%, by weight of the consumer product.

The precise nature of these additional components, and levels of incorporation thereof, will depend on the physical form of the composition and the nature of the operation for which it is to be used. Suitable adjunct materials include, but are not limited to, polymers, for example cationic polymers, surfactants, builders, chelating agents, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' cleaning and laundry care compositions. Thus, certain embodiments of Applicants' compositions do not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids and/or pigments. However, when one or more adjuncts are present, such one or more adjuncts may be present as detailed below:

Surfactants—The compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants. The surfactant is typically present at a level of from about 0.1%, from about 1%, or even from about 5% by weight of the cleaning compositions to about 99.9%, to about 80%, to about 35%, or even to about 30% by weight of the cleaning compositions.

Builders—The compositions of the present invention can comprise one or more detergent builders or builder systems. When present, the compositions will typically comprise at least about 1% builder, or from about 5% or 10% to about 80%, 50%, or even 30% by weight, of said builder. Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is a cocktail of conventional applicable enzymes like protease, lipase, cutinase and/or cellulase in conjunction with amylase.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methyl-enephos [symbol] phonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282.

Cobalt bleach catalysts useful herein are known, and are described, for example, in U.S. Pat. Nos. 5,597,936 and 5,595,967. Such cobalt catalysts are readily prepared by known procedures, such as taught for example in U.S. Pat. Nos. 5,597,936, and 5,595,967.

Compositions herein may also suitably include a transition metal complex of a macropolycyclic rigid ligand—abbreviated as “MRL”. As a practical matter, and not by way of limitation, the compositions and cleaning processes herein can be adjusted to provide on the order of at least one part per hundred million of the benefit agent MRL species in the aqueous washing medium, and may provide from about 0.005 ppm to about 25 ppm, from about 0.05 ppm to about 10 ppm, or even from about 0.1 ppm to about 5 ppm, of the MRL in the wash liquor.

Suitable transition-metals in the instant transition-metal bleach catalyst include manganese, iron and chromium. In one aspect, suitable MRL's herein are a special type of ultra-rigid ligand that is cross-bridged such as 5,12-diethyl-1,5,8,12-tetraazabicyclo[6.6.2]hexa-decane. Suitable transition metal MRLs are readily prepared by known procedures, such as taught for example in WO 00/32601, and U.S. Pat. No. 6,225,464.

### Processes of Making Laundry Care Compositions

The laundry care compositions of the present invention can be formulated into any suitable form and prepared by any process chosen by the formulator, non-limiting examples of which are described in U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; and U.S. Pat. No. 5,486,303.

In one aspect, the liquid detergent compositions disclosed herein may be prepared by combining the components thereof in any convenient order and by mixing, e.g., agitating, the resulting component combination to form a phase stable liquid detergent composition. In one aspect, a liquid matrix is formed containing at least a major proportion, or even substantially all, of the liquid components, e.g., nonionic surfactant, the non-surface active liquid carriers and other optional liquid components, with the liquid components being thoroughly admixed by imparting shear agitation to this liquid combination. For example, rapid stirring with a mechanical stirrer may usefully be employed. While shear agitation is maintained, substantially all of any anionic surfactant and the solid ingredients can be added. Agitation of the mixture is continued, and if necessary, can be increased at this point to form a solution or a uniform dispersion of insoluble solid phase particulates within the liquid phase. After some or all of the solid-form materials have been added to this agitated mixture, particles of any enzyme material to be included, e.g., enzyme prills, are incorporated. As a variation of the composition preparation procedure described above, one or more of the solid components may be added to the agitated mixture as a solution or slurry of particles premixed with a minor portion of one or more of the liquid components. After addition of all of the composition components, agitation of the mixture is continued for a period of time sufficient to form compositions having the requisite viscosity and phase stability characteristics. Frequently this will involve agitation for a period of from about 30 to 60 minutes.

Various techniques for forming detergent compositions in such solid forms are well known in the art and may be used herein. In one aspect, when the laundry care composition is in the form of a granular particle, optionally including additional but not all components of the laundry detergent composition.

### Methods of Using Laundry Care Compositions

The laundry care compositions disclosed in the present specification may be used to clean or treat a fabric. Typically at least a portion of the fabric is contacted with an embodiment of the aforementioned laundry care compositions, in neat form or diluted in a liquor, for example, a wash liquor and then the fabric may be optionally washed and/or rinsed. In one aspect, a fabric is optionally washed and/or rinsed, contacted with a an embodiment of the aforementioned laundry care compositions and then optionally washed and/or rinsed. For purposes of the present invention, washing includes but is not limited to, scrubbing, and mechanical agitation. The fabric may comprise most any fabric capable of being laundered or treated.

The laundry care compositions disclosed in the present specification can be used to form aqueous washing solutions for use in the laundering of fabrics. Generally, an effective amount of such compositions is added to water, for example, in a conventional fabric laundering automatic washing machine, to form such aqueous laundering solutions. The aqueous washing solution so formed is then contacted, in one aspect, under agitation, with the fabrics to be laundered therewith. An effective amount of the laundry care composition, such as the liquid detergent compositions disclosed in the present specification, may be added to water to form aqueous laundering solutions that may comprise from about 500 to about 7,000 ppm or even from about 1,000 to about 3,000 pm of laundry care composition.

In one aspect, the laundry care compositions may be employed as a laundry additive, a pre-treatment composition and/or a post-treatment composition.

### Test Methods

1.) ClogP values: For purposes of the present invention ClogP values are calculated ClogP values. Such values may be obtained from the SciFiner database by American Chemical Society through Chemical Abstract Services (CAS), P.O. Box 3102, Columbus, Ohio 43210. If a ClogP value is not available from SciFiner, the value is calculated in accordance with the fragment approach in Hansch and Leo (cf., A. Leo, in comprehensive medicinal chemistry, Vol.4, C. Hansch, P. G. Sammens, J. B. Taylor and C. A. Ransden, Eds., P. 295, Pergamon Press, 1990,
2.) Boiling Point: For purposes of the present invention the boiling points of perfume materials may be obtained from the SciFiner database by American Chemical Society through Chemical Abstract Services (CAS), P.O. Box 3102, Columbus, Ohio 43210. If a ClogP value is not available from SciFiner, the value is calculated in accordance with the protocol found in “Computer-Assisted Prediction of Normal Boiling Points of Furans, Tetrahydrofurans, and Thiophenes,” D. T. Stanton et al, J. Chem. Inf. Comput. Sci., 31 (1992), pp. 301-310.

### EXAMPLES

The following examples illustrate the compositions of the present invention but are not necessarily meant to limit or otherwise define the scope of the invention herein.

### Example 1

The following neat perfume compositions are made

| | | | | | PRM |
|---|---|---|---|---|---|
| Perfume Materials | Example A | Example B | Example C | Example F | Type |
| Ethyl-2-methyl butyrate | 5.0 | 10.0 | 0.5 | 1.0 | Table 1 |
| Anisic Aldehyde | 5.0 | 10.0 | 0.0 | 0.5 | Table 1 |
| Benzyl acetate | 11.0 | 10.0 | 0.5 | 10.0 | Table 1 |
| Hexyl Acetate | 0.0 | 2.0 | 1.0 | 5.0 | Table 2 |
| Myrcene | 2.0 | 2.5 | 1.0 | 5.0 | Table 2 |
| Linalyl Acetate | 0.5 | 20.0 | 10.0 | 20.0 | Table 2 |
| Citronellol | 2.0 | 1.0 | 10.0 | 15.0 | Table 2 |
| Vertenex | 0.5 | 5.0 | 15.0 | 20.0 | Table 2 |
| Geranyl Nitrile | 0.0 | 1.0 | 4.0 | 5.0 | Table 2 |
| Cedrol | 10.0 | 5.0 | 3.5 | 0.5 | Table 3 |
| Cymal | 10.0 | 5.0 | 10.5 | 0.0 | Table 3 |
| Gamma Methyl Ionone | 10.0 | 5.0 | 10.0 | 0.5 | Table 3 |
| Hexyl Cinnamic | 24.0 | 15.0 | 15.0 | 7.5 | Other |
| Aldehyde | | | | | |
| Methyl | 20.0 | 8.5 | 19.0 | 10.0 | Other |
| dihydrojasmonate | | | | | |
| Sum | 100.0 | 100.0 | 100.0 | 100.0 | |

### Example 2

The following perfume compositions are made and are used as a core in the perfume microcapsules.

| | Example | Example | Example | |
|---|---|---|---|---|
| Perfume Materials | A | B | C | Example D |
| Ethyl Vanillin | 0.5 | — | 1 | 2 |
| Phenyl Ethyl Alcohol | 5 | — | 15 | 25 |
| Benzyl Acetate | 1 | — | 15 | 2 |
| Linalool | 10 | 10 | 20 | 25 |
| Linaly Acetate | 11.5 | 15 | 25 | 5 |
| Flor Acetate | 12 | 15 | 1 | 25 |
| Hexyl Cinnamic | 15 | 20 | 5 | 5 |
| Aldehyde | | | | |
| Benzyl Salicylate | 10 | 5 | 1 | |
| Methyl | 20 | 10 | 10 | 5 |
| Dihydrojasmonate | | | | |
| Gamma Methyl Ionone | 10 | 20 | 5 | 5 |
| Delta Damascone | 5 | 5 | 2 | 1 |
| Sum | 100 | 100 | 100 | 100 |

### Example 3

The following perfume compositions are made and are used as the perfume associated or loaded into or absorbed into the polymer assisted delivery matrix system.

| | | | | | PRM |
|---|---|---|---|---|---|
| Perfume Materials | Example A | Example B | Example C | Example D | Type |
| Ethyl-2-methyl butyrate | 5.0 | 10.0 | 0.5 | 13.0 | Table 1 |
| Anisic Aldehyde | 5.0 | 10.0 | 0.0 | 3.0 | Table 1 |
| Benzyl acetate | 11.0 | 10.0 | 0.5 | 2.0 | Table 1 |
| Hexyl Acetate | 0.0 | 2.0 | 1.0 | 9.0 | Table 2 |
| Myrcene | 2.0 | 2.5 | 1.0 | 8.0 | Table 2 |
| Linalyl Acetate | 0.5 | 20.0 | 10.0 | 3.0 | Table 2 |
| Citronellol | 2.0 | 1.0 | 10.0 | 5.0 | Table 2 |
| Vertenex | 0.5 | 5.0 | 15.0 | 3.0 | Table 2 |
| Geranyl Nitrile | 0.0 | 1.0 | 4.0 | 2.0 | Table 2 |
| Cedrol | 10.0 | 5.0 | 3.5 | 0.4 | Table 3 |
| Cymal | 10.0 | 5.0 | 10.5 | 0.2 | Table 3 |
| Gamma Methyl Ionone | 10.0 | 5.0 | 10.0 | 0.1 | Table 3 |
| Hexyl Cinnamic | 24.0 | 15.0 | 15.0 | 0.2 | Other |
| Aldehyde | | | | | |
| Methyl | 20.0 | 8.5 | 19.0 | 0.3 | Other |
| dihydrojasmonate | | | | | |
| Other | Balance | Balance | Balance | Balance | Tables |
| | | | | | 1-3 |

### Example 4

### Melamine Based Polyurea Capsule (85% core/15% wall)

A first mixture is prepared by combining 208 grams of water and 5 grams of alkyl acrylate-acrylic acid copolymer (Polysciences, Inc. of Warrington, Pa., USA). This first mixture is adjusted to pH 5.0 using sodium hydroxide.

178 grams of the capsule core material which comprise a fragrance oil of Example 2 A, B, C or D is added to the first mixture at a temperature of 65° C. to form an emulsion. High speed blending is used to achieve a volume-mean particle size of 15 microns. The ingredients to form the capsule wall material are prepared as follows: 9 grams of a corresponding capsule wall material copolymer pre-polymer (butylacrylate-acrylic acid copolymer) and 90 grams of water are combined and adjusted to pH 5.0. To this mixture is added 28 grams of a partially methylated methylol melamine resin solution (“Cymel 385”, 80% solids, Cytec). This mixture is added to the above described fragrance oil-in-water emulsion with stirring at a temperature of 65 degrees Centigrade. The temperature of the mixture is maintained at this temperature for 8 hours with continuous stirring to initiate and complete encapsulation.

To form the acrylic acid-alkyl acrylate copolymer capsule wall, the alkyl group can be selected from ethyl, propyl, butyl, amyl, hexyl, cyclohexyl, 2-ethylhexyl, or other alkyl groups having from one to about sixteen carbons, or even one to eight carbons.

### Example 5

### Fabric Conditioning Compositions

The following are non-limiting examples of the fabric conditioning compositions of the present invention.

| Ingredients | Example A | Example B | Example C | Example D |
|---|---|---|---|---|
| FSA^{a} | 14-16.5 | 14-16.5 | 14-16.5 | 14-16.5 |
| Ethanol | 2.2-2.6 | 2.2-2.6 | 2.2-2.6 | 2.2-2.6 |
| Starch^{b} | 1.25-1.5 | 1.25-1.5 | 1.25-1.5 | 1.25-1.5 |
| Neat Perfume | 0.4-1.5 A | 0.4-1.5 B | 0.4-1.5 C | 0.4-1.5 D |
| From Example 1 | | | | |
| Encapsulated Perfume from | 0.6 A | 0.6 B | 0.6 C | 0.6 D |
| Example 2 | | | | |
| Phase Stabilizing Polymer^{c} | 0.14-0.21 | 0.14-0.21 | 0.14-0.21 | 0.14-0.21 |
| Calcium Chloride | 0.1-0.3 | 0.1-0.3 | 0.1-0.3 | 0.1-0.3 |
| DTPA^{d} | 0.017 | 0.017 | 0.017 | 0.017 |
| Preservative (ppm)^{e} | 5 | 5 | 5 | 5 |
| Antifoam^{f} | 0.015 | 0.018 | 0.015 | 0.015 |
| Dye (ppm) | 30-300 | 30-300 | 30-300 | 30-300 |
| Ammonium Chloride | 0.02-0.12 | 0.02-0.12 | 0.02-0.12 | 0.02-0.12 |
| HCl | 0.012 | 0.014 | 0.012 | 0.012 |
| Structurant^{g} | 0.01 | 0.01 | 0.01 | 0.01 |
| Deionized Water | Balance | Balance | Balance | Balance |

^{a}N,N-di(tallowoyloxyethyl)-N,N-dimethylammonium chloride.
^{b}Cationic high amylose maize starch available from National Starch under the trade name CATO ®.
^{c}Copolymer of ethylene oxide and terephthalate having the formula described in U.S. Pat. No. 5,574,179 at col.15, lines 1-5, wherein each X is methyl, each n is 40, u is 4, each R1 is essentially 1,4-phenylene moieties, each R2 is essentially ethylene, 1,2-propylene moieties, or mixtures thereof.
^{d}Diethylenetriaminepentaacetic acid.
^{e}KATHON ® CG available from Rohm and Haas Co. “PPM” is “parts per million.”
^{f}Silicone antifoam agent available from Dow Corning Corp. under the trade name DC2310.
^{g}Hydrophobically-modified ethoxylated urethane available from Rohm and Haas under the tradename Aculan 44.

### Example 6

### Fabric Conditioning Compositions

The following are non-limiting examples of the fabric conditioning compositions of the present invention.

| Ingredients | A | B | C | D | E | F | G |
|---|---|---|---|---|---|---|---|
| FSA^{a} | 14 | 16 | 14 | 18 | 16 | 14 | 14 |
| Ethanol | 2 | 3 | 2 | 4 | 3 | 2 | 2 |
| Starch^{b} | 1.2 | 2.0 | 1.7 | 2.4 | 2.0 | 1.7 | 1.2 |
| Neat Perfume From Example 1A | 0.4 | 0.5 | 0.65 | 1.0 | 0.5 | 0.65 | 0.4 |
| Encapsulated Perfume from Example 2B | 0.6 | 0.5 | 0.7 | 0.8 | 0.5 | 0.7 | 0.6 |
| Phase Stabilizing Polymer^{c} | 0.20 | 0.14 | 0.17 | 0.21 | 0.14 | 0.17 | 0.20 |
| Calcium Chloride | 0.11 | 0.15 | 0.17 | 0.31 | 0.15 | 0.17 | 0.11 |
| DTPA^{d} | 0.017 | 0.015 | 0.019 | 0.019 | 0.015 | 0.019 | 0.017 |
| Preservative (ppm)^{e} | 5 | 5 | 5 | 5 | 5 | 5 | 5 |
| Antifoam^{f} | 0.015 | 0.018 | 0.015 | 0.015 | 0.018 | 0.015 | 0.015 |
| Dye (ppm) | 30 | 80 | 150 | 300 | 80 | 150 | 30 |
| Ammonium Chloride | 0.02 | 0.07 | 0.10 | 0.12 | 0.07 | 0.10 | 0.02 |
| HCl | 0.012 | 0.014 | 0.012 | 0.012 | 0.014 | 0.012 | 0.012 |
| Structurant^{g} | 0.01 | 0.01 | 0.01 | 0.01 | 0.01 | 0.01 | 0.01 |
| Polyethyleneimine (25,000 MW average) | 0.20 | 0.25 | 0.28 | 0.17 | — | — | — |
| Additional Neat Perfume | | | | | | 0.05 | 0.08 |
| Deionized Water* | | | | | | | |
| PAD matrix in the form of a Hot Melt** | — | Yes | — | Yes | — | Yes | — |
| with Loaded Perfume from Example 3A | | | | | | | |

*Balance
**The products above are packaged in a package comprising a container comprising a cap. The packaging in one aspect comprises the aforementioned PAD matrix system in the form of a Hot Melt adhesive. The PAD in form of hot melt adhesive in above examples is placed under or in close proximity to said cap.

In the detergent compositions, the abbreviated component identifications have the following meanings:
LAS: Sodium linear C₁₁-C₁₃ alkyl benzene sulfonate
TAS: Sodium tallow alkyl sulfate
CxyAS: Sodium C₁ₓ-C_{1y} alkyl sulfate
C46SAS: Sodium C₁₄-C16 secondary (2,3) alkyl sulfate
CxyEzS: Sodium C1x-C1y alkyl sulfate condensed with z moles of ethylene oxide
CxyEz: C1x-C1y predominantly linear primary alcohol condensed with an average of z moles of ethylene oxide
QAS: R2.N+(CH₃)₂(C₂H₄OH) with R2=C₁₂-C₁₄
QAS 1: R2.N+(CH₃)₂(C₂H₄OH) with R2=C₈-C₁₁
APA: C₈-C₁₀ amido propyl dimethyl amine
Soap: Sodium linear alkyl carboxylate derived from an 80/20 mixture of tallow and coconut fatty acids
STS: Sodium toluene sulphonate
CFAA: C₁₂-C₁₄ (coco) alkyl N-methyl glucamide
TFAA: C₁₆-C₁₈ alkyl N-methyl glucamide
TPKFA: C₁₂-C₁₄ topped whole cut fatty acids
STPP: Anhydrous sodium tripolyphosphate
TSPP: Tetrasodium pyrophosphate
Zeolite A: Hydrated sodium aluminosilicate of formula Na₁₂(AlO₂SiO₂)₁₂.27H₂O having a primary particle size in the range from 0.1 to 10 micrometers (weight expressed on an anhydrous basis)
NaSKS-6: Crystalline layered silicate of formula δ-Na₂Si₂O₅
Citric acid: Anhydrous citric acid
Borate: Sodium borate
Carbonate: Anhydrous sodium carbonate with a particle size between 200 μm and 900 μm
Bicarbonate: Anhydrous sodium bicarbonate with a particle size distribution between 400 μm and 1200 μm
Silicate: Amorphous sodium silicate (SiO₂:Na₂O=2.0:1)
Sulfate: Anhydrous sodium sulfate
Mg sulfate: Anhydrous magnesium sulfate
Citrate Tri-sodium citrate dihydrate of activity 86.4% with a particle size distribution between 425 μm and 850 μm
MA/AA: Copolymer of 1:4 maleic/acrylic acid, average molecular weight about 70,000
MA/AA (1): Copolymer of 4:6 maleic/acrylic acid, average molecular weight about 10,000
AA: Sodium polyacrylate polymer of average molecular weight 4,500
CMC: Sodium carboxymethyl cellulose
Cellulose ether: Methyl cellulose ether with a degree of polymerization of 650 available from Shin Etsu Chemicals
Protease: Proteolytic enzyme, having 3.3% by weight of active enzyme, sold by NOVO Industries A/S under the tradename Savinase
Protease I: Proteolytic enzyme, having 4% by weight of active enzyme, as described in WO 95/10591, sold by Genencor Int. Inc.
Alcalase: Proteolytic enzyme, having 5.3% by weight of active enzyme, sold by NOVO Industries A/S
Cellulase: Cellulytic enzyme, having 0.23% by weight of active enzyme, sold by NOVO Industries A/S under the tradename Carezyme
Amylase: Amylolytic enzyme, having 1.6% by weight of active enzyme, sold by NOVO Industries A/S under the tradename Termamyl 120T
Lipase: Lipolytic enzyme, having 2.0% by weight of active enzyme, sold by NOVO Industries A/S under the tradename Lipolase
Lipase (1) Lipolytic enzyme, having 2.0% by weight of active enzyme, sold by NOVO Industries A/S under the tradename Lipolase Ultra
Endolase: Endoglucanase enzyme, having 1.5% by weight of active enzyme, sold by NOVO Industries A/S
PB4: Sodium perborate tetrahydrate of nominal formula NaBO₂.3H₂O.H₂O₂
PB1: Anhydrous sodium perborate bleach of nominal formula NaBO₂.H₂O₂
Percarbonate: Sodium percarbonate of nominal formula 2Na₂CO₃.3H₂O₂
NOBS: Nonanoyloxybenzene sulfonate in the form of the sodium salt
NAC-OBS: (6-nonamidocaproyl) oxybenzene sulfonate
TAED: Tetraacetylethylenediamine
DTPA: Diethylene triamine pentaacetic acid
DTPMP: Diethylene triamine penta (methylene phosphonate), marketed by Monsanto under the Tradename Dequest 2060
EDDS: Ethylenediamine-N,N′-disuccinic acid, (S,S) isomer in the form of its sodium salt.
Photoactivated: Sulfonated zinc phthlocyanine encapsulated in
bleach (1) dextrin soluble polymer
Photoactivated: Sulfonated alumino phthlocyanine encapsulated in
bleach (2) dextrin soluble polymer
Brightener 1: Disodium 4,4′-bis(2-sulphostyryl)biphenyl
Brightener 2: Disodium 4,4′-bis(4-anilino-6-morpholino-1.3.5-triazin-2-yl)amino)stilbene-2:2′-disulfonate
HEDP: 1,1-hydroxyethane diphosphonic acid
PEGx: Polyethylene glycol, with a molecular weight of x (typically 4,000)
PEO: Polyethylene oxide, with an average molecular weight of 50,000
TEPAE: Tetraethylenepentaamine ethoxylate
PVI: Polyvinyl imidosole, with an average molecular weight of 20,000
PVP: Polyvinylpyrolidone polymer, with an average molecular weight of 60,000
PVNO: Polyvinylpyridine N-oxide polymer, with an average molecular weight of 50,000
PVPVI: Copolymer of polyvinylpyrolidone and vinylimidazole, with an average molecular weight of 20,000
QEA: bis((C₂H₅O)(C₂H4O)n)(CH₃) —N+-C₆H₁₂—N+-(CH₃) bis((C₂H₅O)—(C₂H4O))n, wherein n =from 20 to 30
SRP 1: Anionically end capped poly esters
SRP 2: Diethoxylated poly (1, 2 propylene terephtalate) short block polymer
PEI: Polyethyleneimine
Silicone antifoam: Polydimethylsiloxane foam controller with siloxane-oxyalkylene copolymer as dispersing agent with a ratio of said foam controller to said dispersing agent of 10:1 to 100:1
Opacifier: Water based monostyrene latex mixture, sold by BASF Aktiengesellschaft under the tradename Lytron 621
Wax: Paraffin wax
DEQA: Di-(tallow-oxy-ethyl) dimethyl ammonium chloride.
DEQA (2): Di-(soft-tallowyloxyethyl) hydroxyethyl methyl ammonium methylsulfate.
DTDMAMS: Ditallow dimethyl ammonium methylsulfate.
SDASA: 1:2 ratio of stearyldimethyl amine:triple-pressed stearic acid.
PA30: Polyacrylic acid of average molecular weight of between about 4,500-8,000.
480N: Random copolymer of 7:3 acrylate/methacrylate, average molecular weight about 3,500.
Polygel/carbopol: High molecular weight crosslinked polyacrylates.
Metasilicate: Sodium metasilicate (SiO₂:Na₂O ratio=1.0).
Nonionic: C₁₃-C₁₅ mixed ethoxylated/propoxylated fatty alcohol with an average degree of ethoxylation of 3.8 and an average degree of propoxylation of 4.5.
Neodol 45-13: C₁₄-C₁₅ linear primary alcohol ethoxylate, sold by Shell Chemical CO.
MnTACN: Manganese 1,4,7-trimethyl-1,4,7-triazacyclononane.
PAAC: Pentaamine acetate cobalt(III) salt.
Paraffin: Paraffin oil sold under the tradename Winog 70 by Wintershall.
NaBz: Sodium benzoate.
BzP: Benzoyl Peroxide.
SCS: Sodium cumene sulphonate.
BTA: Benzotriazole.
pH: Measured as a 1% solution in distilled water at 20° C.

### Example 7

The following high density granular laundry detergent compositions A to F are prepared in accord with the invention:

| Ingredients | Example A | Example B | Example C | Example D |
|---|---|---|---|---|
| Blown powder | | | | |
| LAS | 6.0 | 5.0 | 11.0 | 6.0 |
| TAS | 2.0 | — | — | 2.0 |
| Zeolite A | 24.0 | — | — | 20.0 |
| STPP | — | 27.0 | 24.0 | — |
| Sulfate | 4.0 | 6.0 | 13.0 | — |
| MA/AA | 1.0 | 4.0 | 6.0 | 2.0 |
| Silicate | 1.0 | 7.0 | 3.0 | 3.0 |
| CMC | 1.0 | 1.0 | 0.5 | 0.6 |
| Brightener 1 | 0.2 | 0.2 | 0.2 | 0.2 |
| Silicone | 1.0 | 1.0 | 1.0 | 0.3 |
| antifoam | | | | |
| DTPMP | 0.4 | 0.4 | 0.2 | 0.4 |
| Spray on | | | | |
| Brightener | 0.02 | — | — | 0.02 |
| C45E7 | — | — | — | 5.0 |
| C45E2 | 2.5 | 2.5 | 2.0 | — |
| C45E3 | 2.6 | 2.5 | 2.0 | — |
| Neat Perfume | 0.5 A | 0.3 B | 0.5 C | 0.2 D |
| From | | | | |
| Example 1 | | | | |
| Silicone | 0.3 | 0.3 | 0.3 | — |
| antifoam | | | | |
| Dry additives | | | | |
| QEA | — | — | — | 1.0 |
| EDDS | 0.3 | — | — | — |
| Sulfate | 2.0 | 3.0 | 5.0 | 10.0 |
| Carbonate | 6.0 | 13.0 | 15.0 | 14.0 |
| Citric acid | 2.5 | — | — | 2.0 |
| QAS II | 0.5 | — | — | 0.5 |
| SKS-6 | 10.0 | — | — | — |
| Percarbonate | 18.5 | — | — | — |
| PB4 | — | 18.0 | 10.0 | 21.5 |
| TAED | 2.0 | 2.0 | — | 2.0 |
| NAC-OBS | 3.0 | 2.0 | 4.0 | — |
| Protease | 1.0 | 1.0 | 1.0 | 1.0 |
| Lipase | — | 0.4 | — | 0.2 |
| Lipase (1) | 0.4 | — | 0.4 | — |
| Amylase | 0.2 | 0.2 | 0.2 | 0.4 |
| Brightener 1 | 0.05 | — | — | 0.05 |
| Encapsulated | 0.1 A | 0.3 B | 0.15 C | 0.4 D |
| Perfume of | | | | |
| Example 2 | | | | |
| Misc/minor to | | | | |
| 100% | | | | |

### Example 8

### Heavy Duty Liquid Detergent Composition

The following liquid detergent formulations are prepared in accord with the invention (levels are given as parts per weight).

| | | Example | Example | Example |
|---|---|---|---|---|
| Ingredients | Example A | B | C | D |
| LAS | 11.5 | 8.8 | — | 3.9 |
| C25E2.5S | — | 3.0 | 18.0 | — |
| C45E2.25S | 11.5 | 3.0 | — | 15.7 |
| C23E9 | — | 2.7 | 1.8 | 2.0 |
| C23E7 | 3.2 | — | — | — |
| CFAA | — | — | 5.2 | — |
| TPKFA | 1.6 | — | 2.0 | 0.5 |
| Citric acid (50%) | 6.5 | 1.2 | 2.5 | 4.4 |
| Calcium formate | 0.1 | 0.06 | 0.1 | — |
| Sodium formate | 0.5 | 0.06 | 0.1 | 0.05 |
| Sodium cumene | 4.0 | 1.0 | 3.0 | 1.18 |
| sulfonate | | | | |
| Borate | 0.6 | — | 3.0 | 2.0 |
| Sodium hydroxide | 5.8 | 2.0 | 3.5 | 3.7 |
| Ethanol | 1.75 | 1.0 | 3.6 | 4.2 |
| 1,2 propanediol | 3.3 | 2.0 | 8.0 | 7.9 |
| Monoethanolamine | 3.0 | 1.5 | 1.3 | 2.5 |
| TEPAE | 1.6 | — | 1.3 | 1.2 |
| Protease | 1.0 | 0.3 | 1.0 | 0.5 |
| Lipase | — | — | 0.1 | — |
| Cellulase | — | — | 0.1 | 0.2 |
| Amylase | — | — | — | 0.1 |
| SRP1 | 0.2 | — | 0.1 | — |
| DTPA | — | — | 0.3 | — |
| PVNO | — | — | 0.3 | — |
| Neat Perfume | 0.4 | 0.4 | 0.4 | 0.4 |
| Encapsulated | 0.2 A | 0.5 B | 0.1 C | 0.3 D |
| Perfume of | | | | |
| Example 2 | | | | |
| Brightener 1 | 0.2 | 0.07 | 0.1 | — |
| Silicone antifoam | 0.04 | 0.02 | 0.1 | 0.1 |
| Water/minors | | | | |
| PAD matrix Hot Melt | — | — | Yes | — |
| perfume delivery | | | | |
| system | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product comprising:
a.) from about 0.01% to about 20% of a neat perfume comprising, based on weight of said neat perfume:
(i) from about 1% to about 30% of a perfume raw material having a boiling point less than or equal to 250° C. and a ClogP less than or equal to 2.5;
(ii) from about 5% to about 70% of a perfume raw material having boiling point less than or equal to 250° C. and a ClogP greater than 2.5;
(iv) from about 1% to about 30% of a perfume raw material having boiling point greater than 250° C. but less than or equal to 280° C.; and
b.) from about 0.01% to about 20% of a perfume delivery system; and
c.) the balance of said consumer product being a consumer product ingredient.

2. The consumer product of claim 1 wherein:
a.) said perfume raw material having a boiling point less than or equal to 250° C. and a ClogP less than or equal to 2.5 being selected from a Table 1 perfume raw materials and mixtures thereof;
b.) said perfume raw material having boiling point less than or equal to 250° C. and a ClogP greater than 2.5 being selected from a Table 2 perfume raw materials and mixtures thereof; and
c.) said perfume raw material having boiling point greater than 250° C. but less than or equal to 280° C. being selected from a Table 3 perfume raw materials and mixtures thereof.

3. The consumer product of claim 2:
a.) said perfume raw material having a boiling point less than or equal to 250° C. and a ClogP less than or equal to 2.5 being selected from a Table 1 perfume raw materials numbers 1 through 39 and mixtures thereof;
b.) said perfume raw material having boiling point less than or equal to 250° C. and a ClogP greater than 2.5 being selected from a Table 2 perfume raw materials numbers 1 through 116 and mixtures thereof;
c.) said perfume raw material having boiling point greater than 250° C. but less than or equal to 280° C. being selected from a Table 3 perfume raw materials numbers 1 through 58 and mixtures thereof.

4. The consumer product of claim 3:
a.) said perfume raw material having a boiling point less than or equal to 250° C. and a ClogP less than or equal to 2.5 being selected from a Table 1 perfume raw materials numbers 1 through 29 and mixtures thereof;
b.) said perfume raw material having boiling point less than or equal to 250° C. and a ClogP greater than 2.5 being selected from a Table 2 perfume raw materials numbers 1 through 79 and mixtures thereof;
c.) said perfume raw material having boiling point greater than 250° C. but less than or equal to 280° C. being selected from a Table 3 perfume raw materials numbers 1 through 39 and mixtures thereof.

5. The consumer product of claim 1 wherein said perfume delivery system comprises a perfume delivery system selected from the group consisting of: a polymer assisted delivery system, a monomer-assisted delivery system, an amine assisted delivery system, a cyclodextrin system, a starch encapsulated accord system, inorganic carrier system, a pro-perfume, an amine reaction product and mixtures thereof.

6. The consumer product of claim 5, wherein said perfume delivery system comprises a perfume microcapsule.

7. The consumer product of claim 6, wherein said perfume microcapsule comprises, based on total perfume microcapsule weight, from about 50% to about 95%, perfume, at least 50%, of said perfume being a perfume raw material having a ClogP great than or equal to 1 and a boiling point less than or equal to 350° C.

8. The consumer product of claim 2 wherein said perfume delivery system comprises a perfume delivery system selected from the group consisting of: a polymer assisted delivery system, a reservoir system, a monomer-assisted delivery system, an amine assisted delivery system, a cyclodextrin system, a starch encapsulated accord system, inorganic carrier system, a pro-perfume, an amine reaction product and mixtures thereof.

9. The consumer product of claim 8, wherein said perfume delivery system comprises a perfume microcapsule.

10. The consumer product of claim 9, wherein said perfume microcapsule comprises, based on total perfume microcapsule weight, from about 50% to about 95% perfume, at least 50%, of said perfume being a perfume raw material having a ClogP great than or equal to 1 and a boiling point less than or equal to 350° C.

11. The consumer product of claim 3 wherein said perfume delivery system comprises a perfume delivery system selected from the group consisting of: a polymer assisted delivery system, a reservoir system, a monomer-assisted delivery system, an amine assisted delivery system, a cyclodextrin system, a starch encapsulated accord system, inorganic carrier system, a pro-perfume, an amine reaction product and mixtures thereof.

12. The consumer product of claim 11, wherein said perfume delivery system comprises a perfume microcapsule.

13. The consumer product of claim 12, wherein said perfume microcapsule comprises, based on total perfume microcapsule weight, from about 50% to about 95% perfume, at least 50%, of said perfume being a perfume raw material having a ClogP great than or equal to 1 and a boiling point less than or equal to 350° C.

14. The consumer product of claim 4 wherein said perfume delivery system comprises a perfume delivery system selected from the group consisting of: a polymer assisted delivery system, a monomer-assisted delivery system, an amine assisted delivery system, a cyclodextrin system, a starch encapsulated accord system, inorganic carrier system, a pro-perfume, an amine reaction product and mixtures thereof.

15. The consumer product of claim 14, wherein said perfume delivery system comprises a perfume microcapsule.

16. The consumer product of claim 6, wherein said perfume microcapsule comprises, based on total perfume microcapsule weight, from about 50% perfume, at least 50% of said perfume being a perfume raw material having a ClogP great than or equal to 1 and a boiling point less than or equal to 350° C.

17. A consumer product according to claim 1 wherein said perfume delivery system comprises a microcapsule, and an amine assisted delivery system, and the neat perfume comprises:
(i) from about 2% to about 40% of a perfume raw material having a boiling point less than or equal to 250° C. and a ClogP less than or equal to 2.5;
(ii) from about 4% to about 60% of a perfume raw material having boiling point less than or equal to 250° C. and a ClogP greater than 2.5; and
(v) from about 1% to about 20% of a perfume raw material having boiling point greater than 250° C. but less than or equal to 280 0 C.

18. A consumer product according to claim 17 wherein said polyakylamine comprises a polyethyleneamine.

19. A consumer product according to claim 17 wherein said polyakylamine comprises a polyethyleneamine having a weight average molecular weight in daltons of from about 500 to about 5,000,000.

20. A consumer product according to claim 1 comprising a product, a container and packaging a neat perfume, a perfume microcapsule, and a perfume delivery system that comprises a polymer assisted delivery matrix system comprising a perfume comprising:
(i) from about 1% to about 30% of a perfume raw material having a boiling point less than or equal to 250° C. and a ClogP less than or equal to 2.5;
(ii) from about 5% to about 70% of a perfume raw material having boiling point less than or equal to 250° C. and a ClogP greater than 2.5; and
(iii) from about 1% to about 30% of a perfume raw material having boiling point greater than 250° C. but less than or equal to 280° C.;
said neat perfume comprises:
(i) from about 1% to about 30% of a perfume raw material having a boiling point less than or equal to 250° C. and a ClogP less than or equal to 2.5;
(ii) from about 10% to about 90% of a perfume raw material having boiling point less than or equal to 250° C. and a ClogP greater than 2.5; and
(iv) from about 1% to about 30% of a perfume raw material having boiling point greater than 250° C. but less than or equal to 280° C.

21. A consumer product according to claim 20 wherein said polymer assisted delivery matrix system is in either in whole or in part, in communication with the packaged product's packaging and/or the exterior of the packaged product's container.

22. A consumer product according to claim 20 wherein said polymer assisted delivery matrix system is either in whole or in part in communication with the underside of the aforementioned cap.

23. A consumer product comprising a product, a container and packaging a neat perfume, a perfume microcapsule, and a perfume delivery system that comprises an amine assisted delivery system and a polymer assisted delivery matrix system said neat perfume comprising:
(i) from about 2% to about 40% of a perfume raw material having a boiling point less than or equal to 250° C. and a ClogP less than or equal to 2.5;
(ii) from about 4% to about 60% of a perfume raw material having boiling point less than or equal to 250° C. and a ClogP greater than 2.5; and
(iii) from about 1% to about 20% of a perfume raw material having boiling point greater than 250° C. but less than or equal to 280° C.
